(19) United States
(12) Patent Application Publication
(10) Pub. No.: US 2026/0083392 A1
(43) Pub. Date: Mar. 26, 2026

(54) METHOD AND DEVICE FOR DETERMINING ERECTILE FUNCTION

(71) Applicant: PathRight Medical, Inc., Orem, UT (US)

(72) Inventors: Landon Trost, Spanish Fork, UT (US); Jordan Carter, San Jose, CA (US); Nihar Prakash, San Jose, CA (US); Ashby Shaw, Morgan Hill, CA (US); David Faulk, JR., San Jose, CA (US)

(21) Appl. No.: 19/338,536

(22) Filed: Sep. 24, 2025

Related U.S. Application Data
(60) Provisional application No. 63/698,641, filed on Sep. 25, 2024.

(51) Int. Cl. A61B 5/00; A61B 5/053; A61B 5/107

(52) U.S. Cl. CPC A61B 5/4393; A61B 5/002; A61B 5/053; A61B 5/1072; A61B 5/6802; A61B 5/7405; A61B 5/742; A61B 5/6831

(57) ABSTRACT

A method for determining erectile function of an individual includes obtaining, from a wearable device worn on a penis of the individual during stimulation of the penis, one or more displacement values characterizing a measure of displacement of the wearable device by the penis, determining, using the one or more displacement values, an erection quality score of the penis.

### CROSS-REFERENCE TO RELATED APPLICATION

Priority is claimed to U.S. Provisional Patent Application No. 63/698,641, filed Sep. 25, 2024, the entire contents of which are incorporated herein by reference.

### TECHNICAL FIELD

The present disclosure relates to erectile function, and particularly, to a device and method for determining erectile function.

### BACKGROUND

Erectile function plays a crucial role in the process of natural conception and sexual satisfaction. For natural conception to occur, sperm must be delivered to the female reproductive tract through sexual intercourse. Erectile dysfunction (ED) can make it difficult to achieve and maintain an erection sufficient for achieving natural conception and sexual satisfaction. To treat ED, urologists evaluate erectile function by asking ED patients about the frequency or quality of their erections. Patient responses can help tailor treatment for the individual, but success of ED treatment can, in part, depend on the accuracy of these self-evaluations.

### SUMMARY

This disclosure relates to technologies and methods for determining erectile function. In some examples described below, a sensor device is configured for placement around a penis of an individual to actively measure penis circumference, rigidity, or both during sexual intercourse or activity. Data captured by the sensor device is processed to determine a quality score of the erection, and this quality score may be delivered as feedback to a user, such as a wearer of the erectile function device (the individual), a medical professional, and/or other third party. This feedback may be displayed or delivered in visual, audible, or tactile format to inform the individual and/or a healthcare provider of erection trends and performance of the individual over time to create a customized treatment plan for ED.

In accordance with a first aspect of the present disclosure, a method for determining erectile function of an individual may include obtaining, from a wearable device worn on a penis of the individual during stimulation of the penis, one or more displacement values characterizing a measure of displacement of the wearable device by the penis, and determining, using the one or more displacement values, an erection quality score of the penis.

In accordance with a second aspect, a wearable device for quantifying erectile function may include a printed circuit board (PCB) configured to provide an electrical current, a sensor electrically coupled to the PCB, wherein the sensor comprises a ring-shaped body configured for placement around a penis of a user of the wearable device. The body may include an interior surface configured to be placed against a penis of a user of the wearable device, an exterior surface opposite the interior surface, and an encapsulated layered assembly. The layered assembly may include a first layer of conductive material, a second layer of conductive material arranged approximately in parallel with the first layer of conductive material, a dielectric layer disposed between the first and second layers of conductive material, a base layer defining the interior surface of the body, and a top layer defining the exterior surface of the body. A read-out circuit may be coupled to the sensor and may be configured to capture a capacitance value that specifies an ability to store electric charge between at least the first layer of conductive material and the second layer of conductive material.

In accordance with a third aspect, a method communicating a quality of an erection of an individual may include obtaining, from a wearable device worn on a penis of the individual during stimulation of the penis, one or more displacement values characterizing a measure of displacement of the wearable device by the penis. The method may include determining whether the one or more displacement values satisfy a predetermined threshold, and in response to determining that the one or more displacement values satisfy the predetermined threshold, controlling a connected device to perform an operation.

In accordance with a fourth aspect, a method may include obtaining, from a wearable device worn on a penis during stimulation of the penis, one or more displacement values including a first set of values and a second set of values, determining, using the one or more displacement values, a start of an erection session corresponding to the second set of values, and removing, from memory, the first set of values.

In accordance with a fifth aspect, a method of fabricating a layered sensor may include applying a first layer of conductive material on a base layer of elastic material, wherein the first layer of conductive material comprises a first end and a second end opposite the first end, wherein the first end comprises a portion configured to connect with an electrical lead. The method may include applying a layer of dielectric material on the first layer of conductive material, and applying a second layer of conductive material on the layer of dielectric material such that the second layer of conductive material is parallel relative to the first layer of conductive material. The second layer of conductive material may include a portion configured to connect with an electrical lead. The method may include applying a top layer of dielectric material over the second layer of conductive material to form an encapsulated layered assembly, wherein the portion of the second layer of conductive material is offset relative to the portion of the first layer of conductive material about a longitudinal axis of the layered assembly.

In accordance with a fifth aspect, a methods may include the actions of obtaining, from a wearable device worn on a penis of the individual during stimulation of the penis, one or more resistance values, and determining, using the one or more resistance values and a previously stored value indicating a first circumference of the penis, an erection quality score of the penis. Other embodiments of this aspect include corresponding computer systems, apparatus, and computer programs recorded on one or more computer storage devices, each configured to perform the actions of the methods.

In accordance with a sixth aspect, a sensor may include a circuit comprising a first end and a second end, a pathway coupling the first end and the second end of the circuit; and an ionic material disposed in the pathway and configured to carry an electrical current between the first end and the second end of the circuit. The circuit may be configured to detect a change in electrical resistance between the first end and the second end.

In accordance with a seventh aspect, a wearable device may include a printed circuit board (PCB) configured to provide an electrical current and a band electrically coupled to the PCB. The band may include an ionic material that is configured to transmit the electrical current. A circuit may be configured to measure a resistance of the electrical current through the ionic material of the band when the wearable device is worn on a penis of an individual.

In accordance with a eighth aspect, a method may include actions of obtaining, from a wearable device worn on a penis of the individual during stimulation of the penis, one or more resistance values, determining the one or more resistance values satisfy a predetermined threshold, and in response to determining the one or more resistance values satisfy the predetermined threshold, controlling a connected device to perform an operation. Other embodiments of this aspect include corresponding computer systems, apparatus, and computer programs recorded on one or more computer storage devices, each configured to perform the actions of the methods.

In further accordance with any one or more of the first, second, third, fourth, fifth, sixth, seventh, and eighth aspects, the wearable device, sensor, and methods may include any one or more of the following features.

In one example, the wearable device may include a band configured to be worn on the penis, and wherein the method may include obtaining a resistance factor of the band.

In this example, determining the erection quality score may include, using the resistance factor to determine the erection quality score of the penis based on a resistive force received or sensed by the wearable device.

In one example, determining may include using a resistance factor of the band, a maximal circumference of the penis, and the one or more displacement values, an erection quality score of the penis.

In another example, the resistance factor may correspond to a force range of 0.5 N to 10 N.

In one aspect, the method may include transmitting an indication of the erection quality score to a user device.

In another aspect, the method may include generating a feedback signal indicating erection performance.

In some aspects, the erection performance may be calculated using the erection quality score.

In some aspects, the method may include activating the wearable device by generating an activation signal using a user device.

In one aspect, the method may include transmitting the activation signal to the wearable device.

In one example, transmitting the activation signal to the wearable device may include sending a short-range wireless communication to the wearable device.

In some examples, the wearable device may include a capacitance sensing circuit.

In another example, obtaining the one or more displacement values may include obtaining, by the capacitance sensing circuit, one or more capacitance values.

In one example, the method may include transmitting an indication of the erection quality score to one or more user devices in real-time.

In one aspect, the wearable device may include a layered sensor including a first layer of conductive ink and a second layer of conductive ink approximately in parallel with the first layer of conductive material.

In one aspect, a dielectric layer may be between the first and second layers of conductive material.

In another aspect, a top layer may be configured to define an exterior surface of the layered sensor.

In other aspects, a base layer may be configured to define an interior surface of the layered sensor.

In some aspects, obtaining the one or more capacitance values representing the measure of displacement may include obtaining, from the capacitance sensing circuit, a capacitance value that specifies an ability to store electric charge between the first and second layers of conductive material at a displaced state of the wearable device.

In another aspect, the one or more capacitance values may correspond with the displaced state of the layered sensor of the wearable device.

In one example, the layered sensor may include a third layer of conductive material between the first and second layers of conductive material.

In another example, a second dielectric layer may be between the third layer of conductive material and the first layer or the second layer of conductive material.

In another example, the method may include comparing the erection quality score to one or more stored erection quality scores.

In one aspect, the method may include modifying the erection quality score based on one or more correction factors, wherein at least one of the correction factors corresponds with a first circumference of the penis.

In another aspect, the read-out circuit may be coupled to the layered body through analog front-end circuitry connected to the electrical connection.

In other aspects, the dielectric layer may be a first dielectric layer.

In some aspects, the layered body may include a third layer of conductive material.

In another aspect, the layered body may include a second dielectric layer disposed between the second and third layers of conductive material.

In one example, the layered body may include a base layer and a top layer encapsulating the first, second, and third layers of conductive material.

In some examples, the electrical connection may include a first electrical lead coupled to the first and third layers of conductive material.

In one example, the electrical connection may include a second electrical lead coupled to the second layer of conductive material.

In one aspect, one or more of the base layer, the top layer, first dielectric layer, and second dielectric layer may include a silicone material with a first durometer and a second durometer less than the first durometer.

In another aspect, the one or more of the base layer, the top layer, the first dielectric layer, and the second dielectric layer may include a first portion coupled to the electrical connection with the first durometer.

In some aspects, a second portion may include the second durometer.

In other aspects, the conductive material may include a conductive ink.

In other examples, determining the erection quality score of the penis may include determining the erection quality score using a measure of change in displacement of the wearable device at a first displaced state and a second displaced state based on the one or more capacitance values.

In some examples, determining the erection quality score of the penis may include calculating a rigidity of the penis using the one or more displacement values.

In some examples, the method may include determining, using the one or more resistance values, a series of two or more circumference measurements of the penis.

In one aspect, the method may include calculating an average rigidity of the penis using the series of two or more circumference measurements over time.

In another aspect, determining the erection quality score of the penis may include calculating a rigidity of the penis using the one or more resistance values and the previously stored value indicating the first circumference of the penis.

In some aspects, the method may include determining a second circumference of the penis using the one or more resistance values.

In one example, the method may include determining, using the first circumference and the second circumference, the erection quality score.

In some examples, the wearable device may include a band that is configured to be worn on the penis, and wherein actions may include obtaining a resistance factor of the band.

In another example, actions may include determining the erection quality score of the penis using the resistance factor.

In one aspect, actions may include determining the erection quality score of the penis using (i) the resistance factor and (ii) a surface area of the band.

In one aspect, the resistance factor may correspond to a pressure range of 5,000 pascals to 40,000 pascals.

In another aspect, the wearable device may include a tube at least partially filled with an ionic material.

In some aspects, the tube may define a ring that is configured to be worn on the penis.

In one example, obtaining the one or more resistance values may include obtaining one or more resistance values representing a resistance of an electrical current flowing through the ionic material in the tube.

In another example, the wearable device may include a resistance sensing circuit

In another example, obtaining the one or more resistance values may include obtaining one or more resistance values representing a resistance measured by the resistance sensing circuit.

In some examples, actions may include activating the wearable device by generating an activation signal using a user device.

In one aspect, actions may include transmitting the activation signal to the wearable device.

In some aspects, providing the activation signal to the wearable device may include sending a short-range wireless communication to the wearable device.

In one example, the pathway may be at least partially defined by an elastic material.

In some examples, the elastic material may define a tube comprising a first tube end and a second tube end.

In some examples, the pathway may extend between the first tube end and the second tube end.

In one aspect, the sensor may include a first electrical lead coupled to the first end of the circuit and a second electrical lead coupled to the second end of the circuit.

In another aspect, the first electrical lead may be coupled to the first tube end and the second electrical lead may be coupled to the second tube end.

In some aspects, the first electrical lead may be configured to transmit the electrical current to the first tube end, and the second electrical lead may be configured to receive the electrical current from the second tube end.

In one example, the elastic material may be silicone.

In another example, the ionic material may include an ionic fluid.

In some examples, the ionic material disposed in the pathway may be configured to electrically couple the first end and the second end of the circuit.

In one aspect, the band may include a tube with a first end, a second end, and a flow path extending between the first end and the second end.

In some aspects, the wearable device may include a housing coupled to the layered sensor, wherein the PCB is disposed in the housing.

In some examples, the electrical connection may include a first lead coupled to the first layer of conductive material and a second lead coupled to the second layer of conductive material.

In one example, the first lead may provide a sensor input to the read-out circuitry and the second lead may provide a reference input to the read-out circuit through front-end circuitry.

In some aspects, the wearable device may include a housing coupled to the band, wherein the PCB is disposed in the housing.

In one example, the wearable device may include an electrical connection comprising a first end coupled to the band and a second end coupled to the housing.

In another example, the electrical connection may be configured to detect an electrical resistance at one or more of the first and second ends of the band.

In some examples, the electrical connection may include a first lead coupled to the first end of the band and a second lead coupled to a second end of the band, wherein the first lead is configured to deliver the electrical current and the second lead is configured to receive a measured electrical current.

In one aspect, the electrical connection may be removably coupled to the housing.

In another aspect, the electrical connection may be fixed to the sensor.

In other aspects, the body of the sensor may include a resistance factor corresponding to a force range of 1 to 5 Newtons.

In some aspects, the read-out circuit may include a capacitance-to-voltage or capacitance-to-current converter.

In some aspects, the conductive material may include one or more of a metallic based conductive ink, a carbon-based conductive ink, a silicone-based conductive ink, or an ionic gel paste-based conductive ink

In another aspect, the electrical connection may be fixed to the band.

In some aspects, the housing may include an adhesive configured to attach the housing to a skin surface of the individual.

In one example, the band may include a material with a resistance factor corresponding to a pressure range of 5,000 pascals to 40,000 pascals.

In another example, controlling the connected device to perform the operation may include controlling a light to change an illumination state.

In some examples, controlling the light to change the illumination state may include changing a color of the light from a first color to a second color.

In one aspect, controlling the light to change the illumination state may include controlling an intensity of the light.

In another aspect, controlling the connected device to perform the operation may include controlling a speaker to emit audio.

In some aspects, controlling the speaker to emit audio may include controlling the speaker to indicate a quality of erection based on the one or more resistance values.

In other aspects, the method may include determining, using the one or more displacement values, an erection quality score of the penis.

In one example, actions may include determining, using the one or more resistance values and a previously stored value indicating a first circumference of the penis, an erection quality score of the penis.

In another example, the method may include comparing the erection quality score to one or more stored erection quality scores.

In some examples, the method may include determining, using the one or more displacement values, an average rigidity of the penis over time.

In some examples, the method may include determining, using the one or more resistance values, a series of two or more circumference measurements of the penis.

In one aspect, the method may include calculating an average rigidity of the penis using the series of two or more circumference measurements over time.

In another aspect, determining the erection quality score of the penis may include calculating a rigidity of the penis using the one or more resistance values and the previously stored value indicating the first circumference of the penis.

In one example, determining the start of the erection session corresponding to the second set of values may include determining the second set of values indicate a rigidity that satisfies a predetermined threshold.

In some cases, determining the start of the erection session corresponding to the second set of values may include determining the second set of values indicate a penis circumference that satisfies a predetermined threshold.

In some aspects, the method may include determining a second circumference of the penis using the one or more resistance values.

In other aspects, the method may include determining, using the first circumference and the second circumference, the erection quality score.

In one example, the method may include bonding a first end of the encapsulated layered assembly to a second end of the encapsulated layered assembly to form a ring-shaped body.

In another example, the method may include coupling an end of a first electrical lead to the first layer of conductive material at the first end of the layered assembly, and an end of a second electrical lead to the second layer of conductive material at the first end of the layered assembly.

In some examples, the method may include overmolding the bonded first and second ends of the ring-shaped body and the ends of the first and second electrical leads to form an overmolded body, wherein the first and second electrical leads extend from the overmolded body.

In other examples, the base layer may include a silicone material with a first portion at a first end of the base layer and a second portion extending from the first portion, wherein the first portion has a first durometer, and the second portion has a second durometer less than the first durometer.

In one example, applying the first layer of conductive material comprises applying the first end of the first layer of conductive material onto the first portion of the base layer, wherein the portion at the first end of the first layer of conductive material terminates in a narrow tab.

In some examples, the layer of dielectric material may include a first portion and a second portion, wherein the first portion has a first durometer, and the second portion has a second durometer less than the first durometer.

In other examples, applying the layer of dielectric material comprises applying the first portion of the layer of dielectric material adjacent to the first end of the first layer of conductive material.

Systems and methods described in the present disclosure can include one or more of the following advantages. The wearable devices described here allow an individual to track and monitor sexual performance in privacy. The wearable devices may be battery powered and worn discreetly without disturbing sexual activity or providing discomfort. According to the teachings of the present disclosure, the device is configured to fit a wide range of individuals having varying girth, length, and rigidity, and to provide erection quality scores that are normalized by girth. In some examples, the device may be adapted to the individual's comfort and unique physiology by swapping out a wearable portion of the device. Using various connections, as described here, the wearable devices may be easily applied to the body, assembled, and/or disassembled for replacement or reuse. Devices may be made light weight and comfortably attached using a reusable, skin-safe adhesive, to further reduce any disruptions to a given sexual activity.

The technologies and methods described here may also capture, process, and display erection data to help create a customized erectile function treatment plan or therapy for the individual. For example, using devices described herein to evaluate erectile function of an individual may help eliminate user-error, inaccuracies, or other bias prevalent in self-evaluating erectile performance. Sensor data captured by wearable sensors may be transmitted and evaluated in real-time by a healthcare provider, thereby delivering raw and reliable data to inform accurate sexual performance and to create a customized treatment plan or therapy. Feedback may be provided to a personal electronic device of the wearer or other person to inform the individuals while also reducing disruptions to sexual activity, e.g., by providing audio, visual, or tactile feedback. In some examples, devices may enable the setting and real-time feedback in accomplishing sexual-related goals, e.g., as part of an erectile treatment plan.

The terms “top,” “bottom,” “upper,” “lower,” “above,” and “below” are used to provide a relative relationship between structures. The use of these terms does not suggest or require that a particular structure must be located at a particular location in the apparatus.

Some examples may be described using the expression “coupled” and “connected” along with their derivatives. For example, some arrangements may be described using the term “coupled” to indicate that two or more elements are in direct physical or electrical contact. The term “coupled,” however, may also mean that two or more elements are not in direct contact with each other, but yet still co-operate or interact with each other.

The examples described here are not limited in this context.

The details of one or more embodiments of the subject matter of this specification are in the drawings and the description below. Other features, aspects, and advantages of the subject matter will become apparent from the description, the drawings, and the claims.

### BRIEF DESCRIPTION OF THE DRAWINGS

FIG. 1 is a front, perspective view of a device for determining erectile function attached to a penis of a human subject;

FIG. 2 is an exploded, top view of the device of FIG. 1;

FIG. 2A is a diagram of internal components of a housing component of the device of FIG. 1;

FIG. 2B is a magnified view of a juncture of the device of FIG. 1;

FIG. 2C is a magnified, internal view of the juncture of FIG. 2B;

FIG. 3 is a system for determining erectile function configured for use with the device of FIG. 1;

FIG. 4A is a flowchart of an example process for determining an erection quality score;

FIG. 4B is a flowchart of an example process for processing resistance values for determining erectile function;

FIG. 4C is a flowchart of an example process for controlling connected devices in response to determining erectile function;

FIG. 5 is a top, exploded view of another example device for determining erectile function;

FIG. 6A is a perspective view of a housing assembly of the device of FIG. 5;

FIG. 6B is an exploded, partial perspective view of the housing assembly of FIG. 6A;

FIG. 7A is a partially transparent, top view of a sensor of the device of FIG. 5;

FIG. 7B is a magnified view of a band of the sensor of FIG. 7A;

FIG. 7C is a cross-sectional view of the sensor taken at C-C of FIG. 7A;

FIG. 8A is an exploded view of a layered assembly of the sensor of FIG. 7A during sensor fabrication;

FIG. 8B is a cross-sectional side view of the layered assembly of the sensor of FIG. 8A;

FIG. 8C is a top view of the layered assembly of FIG. 8B and an electrical connection;

FIG. 9A is another example sensor body of a sensor of the wearable device of FIG. 5;

FIG. 9B is a circuit diagram of the sensor of FIG. 5;

FIG. 9C depicts an equivalent circuit diagram of the circuit of FIG. 9B;

FIG. 10 is a schematic showing how the sensor of FIG. 7A may capture different capacitance values during a relaxed displaced state and an erect displaced state;

FIG. 11 is a system for determining erectile function configured for use with the device of FIG. 5;

FIGS. 12A and 12B depict example results from a layered sensor, demonstrating a relationship between change in capacitance and erection quality score;

FIG. 13A is a perspective view of another example device for determining erectile function;

FIG. 13B is a front view of the device of FIG. 13A;

FIG. 13C is a cross-sectional view of a housing component of the device taken at section B-B of FIG. 13B;

FIG. 13D is a top, transparent view of the housing component of the device of FIG. 13B;

FIG. 14A depicts an example user interface for logging goals that can be tracked using a device for determining erectile function, such as the devices of FIGS. 1, 5, 13A; and

FIG. 14B depicts an example user interface for tracking an erection at a first time and a second, later time during an erection session using a device for determining erectile function, such as the devices of FIGS. 1, 5, 13A.

Like reference numbers and designations in the drawings reflect like elements.

### DETAILED DESCRIPTION

This disclosure relates to technologies and methods for determining erectile function of an individual during intercourse, penis stimulation, or other sexual acts (referred herein as an “sexual activity”). During such activity, a device worn by the individual can monitor, measure, record, store, and transmit data of the individual's erection for processing and determining erectile performance of the individual over time. Erectile performance data may be used to inform a healthcare provider for customizing an ED treatment plan or therapy and set goals and track erectile performance and progress for the individual. As used herein, “erectile function” may include erectile rigidity and the ability to maintain an erection.

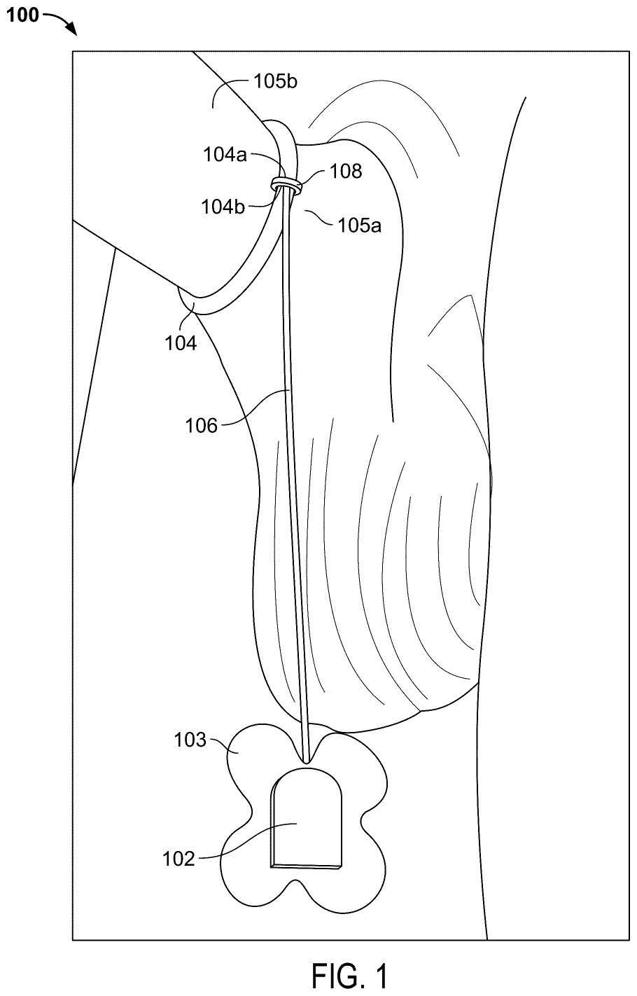

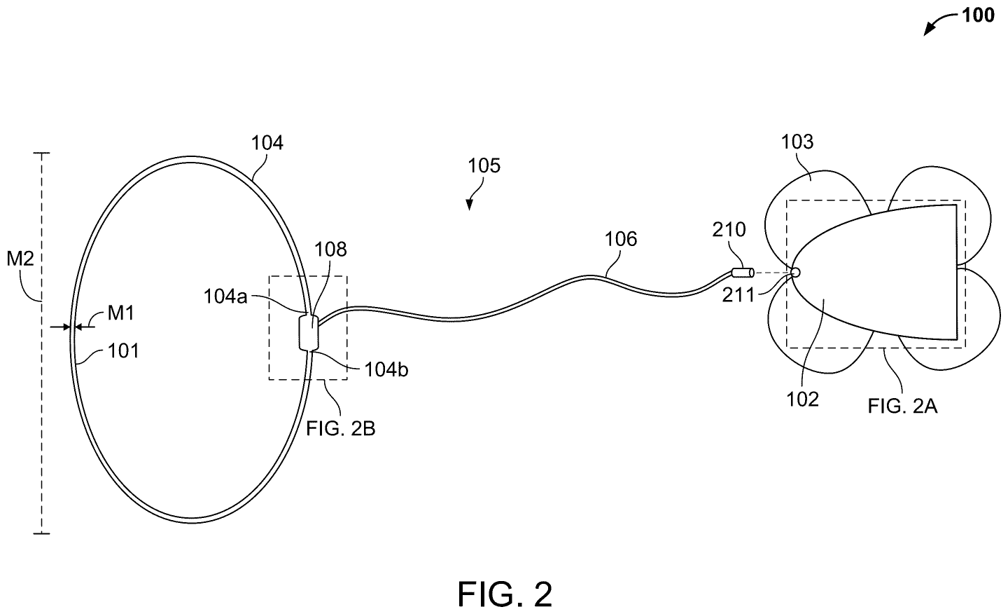

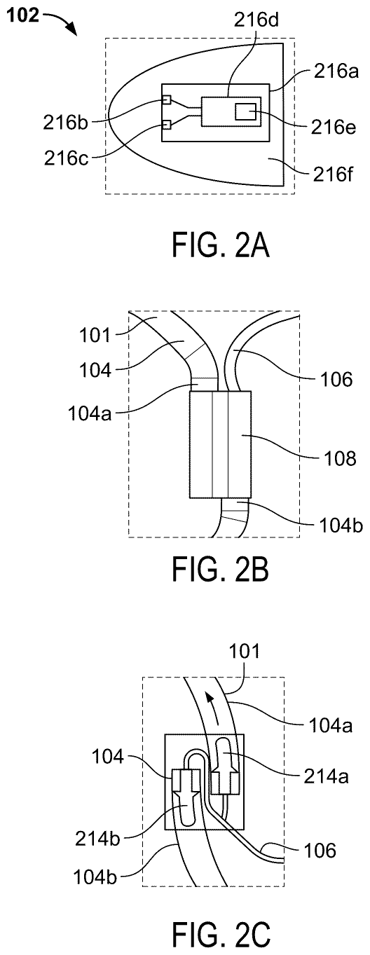

Turning first to FIGS. 1 and 2, a wearable device 100 for determining erectile function is configured to be worn by an individual during sexual activity. The device 100 includes a housing component 102, a band 104, and an electrical connection 106 coupling the band 104 to the housing component 102. The connection 106 includes two lead wires that electrically couple the housing component 102 with the band 104 at a juncture 108.

As shown in FIG. 1, the band 104 is placed around a base 105a of the individual's penis 105b before an erection session begins. The housing component 102 is attachable to the body of the individual so that the housing component 102 does not interfere with or provide discomfort to the individual or sexual partner during, sexual activity. The housing component 102 dangles from the band 104 when the housing component 102 has not yet been attached to the body of the individual. Using an adhesive patch 103 coupled to the housing component 102, the housing component 102 is configured to be removably secured to the individual's perineum, lower abdomen, upper inner thigh, or other location near the individual's pelvis. The patch 103 may be configured for multiple applications, or the patch 103 may be replaceable after each erection session. “Erection session” refers to the time during sexual activity in which the erection data is captured for processing to determine erection quality.

As shown in FIG. 2A, the housing component 102 includes a housing or body 216f (i.e., a housing) that accommodates a printed circuit board (PCB) 216a and a power source 216e coupled to the PCB 216a. The housing 216f is sealably coupled to the electrical connection 106 and sealed from the environment to protect the internal components of the housing component 102 from exposure to external fluids and impact. The PCB 216a is configured to provide electrical current to the device 100 and forms a circuit 216d that measures a resistance of an electrical current through the band 104. For example, as the penis of the individual engorges with blood causing the band 104 to change in circumference, the circuit of the housing component 102 measures the resistance of the electrical current through the band 104. Through a transmitter or other wireless communication component, the PCB 216a transmits this data externally for processing, as elaborated on below.

The circuit board 216a includes electrical connections 216b and 216c that are configured to transfer electrical current to and from the band 104, e.g., via the first juncture 108 and a second juncture 210. Electrical current is generated by the power source 216e, which may include a battery or other form of power. The sensing circuit 216d of the housing component 102 is configured to measure a resistance of the electrical current provided through the band 104 based on the current provided via one of the electrical connections 216b or 216c and current received from the other of the electrical connections 216b and 216c. The sensing circuit 216d or another element of the housing component 102 or connected system may perform further processing based on a detected electrical resistance value, as provided in more detail below. Additional operations may be performed in response to determining one or more aspects of erectile function.

In FIG. 2, the band 104 includes a tube 101 having a first end 104a, a second end 104b, and a flow path (e.g., as shown by the arrow in FIG. 2C) extending between the first and second ends 104a, 104b. As shown in FIGS. 2 and 2B, the first and second ends 104a, 104b are coupled to the housing component 102 via the electric connection 106 at the juncture 108. The first and second ends 104a, 104b, and a terminal end of the electric connection 106 are sealed from the external environment at the juncture 108. The juncture 108 is configured to allow an electrical current to flow from, or to, one or both conductive ends 104a, 104b of the band 104 and from, or to, the housing component 102.

The band 104 creates, in part, a flow path for electrical current, e.g., that extends between the first and second ends 104a, 104b of the band 104. The flow path is defined by the tube 101 that is at least partially filled with an ionic material. In some cases, a width of the tube 101 is fewer than 10 millimeters. The band 104 includes an elastic material (such as, for example, silicone, rubber, or latex). The elastic material can be selected so as not to apply a force great enough to interfere with an erection. For example, the elastic material can be selected to provide a pressure on the penis 105b within a range, such as 5,000 pascals to 40,000 pascals, corresponding to approximately 1 Newton over 200 square millimeters of area to 6 Newtons over 150 square millimeters. The elastic material of the band 104 can provide a force of an area of the band 104 on the penis 105b which can be represented in units of pressure, such as pascals.

Selecting the elastic material can include the type of material, the width of the material, or the thickness of the material for the band 104. The band 104 can have a spring rate such that the pressure applied to the penis 105b is not less than a minimum amount, e.g., to avoid the device 100 slipping off during use, but not more than a maximum amount, e.g., to avoid the device 100 negatively impacting an erection. In some cases, the minimum amount can be approximately 5,000 pascals and the maximum amount can be approximately 40,000 pascals. The pressure applied by the band 104 on the penis 105b can be referred to as a resistance factor of the band 104, where bands with higher resistance factors exert more pressure than bands with lower resistance factors.

An ionic material is disposed in the flow path and is configured to transmit electrical current between the first and second ends 104a, 104b of the tube 101. The ionic material disposed in the flow path is a solution, such as a solution including one or more of sodium chloride magnesium oxide, calcium carbonate, potassium bromide, or aluminum oxide.

The band 104 has a width M1 in a range around 1 mm to approximately 7 mm. The band has a diameter M2 in a range around 15 mm to approximately 60 mm. The electrical connection has a length in a range around 10 mm to approximately 500 mm. In general, thinner bands may be preferable to avoid constriction of the penis. Wider bands may help to avoid slipping off the penis during sexual activity. In cases where the band 104 has dimensions M1 and M2, pressure provided by the band 104 on the penis 105b can range from, approximately, F1/A1 to F2/A2 where F1 and F2 represent a force of an elastic material of the band 104 at a contracted and stretched state, respectively; and A1 and A2 represent area of the contracted and stretched band, respectively. The band 104 may shrink in width as it expands to a larger diameter. A1 can be given by 2πrw or πdw where w is equal to the width of the band and d is equal to the diameter. A1 can be π15 mm*7 mm≈330 mm² and A2 can be π60 mm*1 mm≈190 mm². To keep the pressure applied to the penis 105b in a pressure range P1 to P2, e.g., 5,000 pascals to 40,000 pascals or other suitable range, the elastic material can be configured to provide a force based on the following:

$$F 1 / A 1 = P 1 ; F 2 / A 2 = P 2.$$

$$For example ,$$

$$F 1 can be 5000 Pa * 330 {mm}^{2} = 1650000 \frac{N}{{m}^{2}} {mm}^{2} = 1.65 Newtons and$$

$$F 2 can be 40000 Pa * 190 {mm}^{2} = 7.6 Newtons .$$

When the band is a tube, surface area S may be instead calculated may be approximated as area of a rectangle (length L*width W), where length L is circumference C of an inner diameter D of the tube, and width W is ½ circumference of the diameter d of the tube wall because only half of the tube is in contact with the body. Accordingly,

$$S = L * W = π D * \frac{1}{2} π d = \frac{{π}^{2} * D * d}{2} .$$

In FIG. 2C, first and second electrical leads 214a and 214b are coupled to the electrical flow path of the band 104. The first electrical lead 214a is coupled to the first end 104a of the tube 101, and the second electrical lead 214b is coupled to the second end 104b of the tube 101. At the band 104, the electrical leads 214a, 214b split to couple to opposite ends 104a, 104b of the tube 101. The electrical leads 214a, 214b are bundled in the electrical connection 106. When the device is in use, electrical current is configured to flow in one direction between the first and second leads 214a, 214b. The housing component 102 is configured to provide a current to one of the electrical leads 214a and 214b and to receive current from the other one of the electrical leads 214a and 214b.

In some examples, the first and second ends 104a, 104b of the band 104 include metallic end caps (such as leads 214a and 214b) that allow electrical current to flow from an ionic material within the band 104 to the housing component 102. End caps can help seal the tube 101, e.g., to substantially contain an ionic fluid. Electrical current transferred by conductive ends is processed by one or more circuits within the housing component 102.

Returning to FIG. 2, a band assembly 105, which includes the band 104 and the electrical connection 106, is separated from the housing component 102. The housing component 102 is detachable from the band assembly 105 at the second juncture 210. The second juncture 210 is electrically coupleable to a port 211 in the housing component 102, and includes an electric connection configured to transmit an electric current between the housing component 102 and the band 104 when coupled to the housing component 102.

The second juncture 210 facilitates connection and disconnection between the band assembly 105 and the housing component 102 to replace either the band assembly 105 or the housing component 102 for use in other erection sessions. The second juncture 210 is configured to allow a user to remove the band assembly 105 from the reusable housing component 102, and replace the used band assembly 105 with a new band assembly (e.g., after one or more uses, after a defined amount of time, when inaccurate readings or errors are detected, after notification by the device 100, or whenever a user chooses). While the housing component 102 is configured to be reused and the band assembly 105 discarded, in other examples, the housing component 102 may be discarded and the band assembly 105 may be reused for other erection sessions. In yet other examples, the entire device may be reused for multiple erection sessions.

In implementations with battery power (e.g., rechargeable or non-rechargeable), the device 100 may warn a user if the user activates or puts on the device 100 and the device 100 does not satisfy a power threshold—e.g., a battery does not include sufficient charge to last for a predicted amount of time based on average sexual activity length or based on prior sexual activity data for a specific individual.

So configured, the wearable device 100 includes elements for providing and sensing an electrical current flow from a current producing circuit, such as the circuit board 216a, and through the band 104 disposed around the individual's penis 105b. The current flows between the first end 104a and the second end 104b, through the juncture 108, and to and from the housing component 102. The housing component 102 receives electrical current measured between the first and second ends 104a, 104b of the band 104 (indicative of change in circumference of the penis) for further processing to determine elements of erectile function.

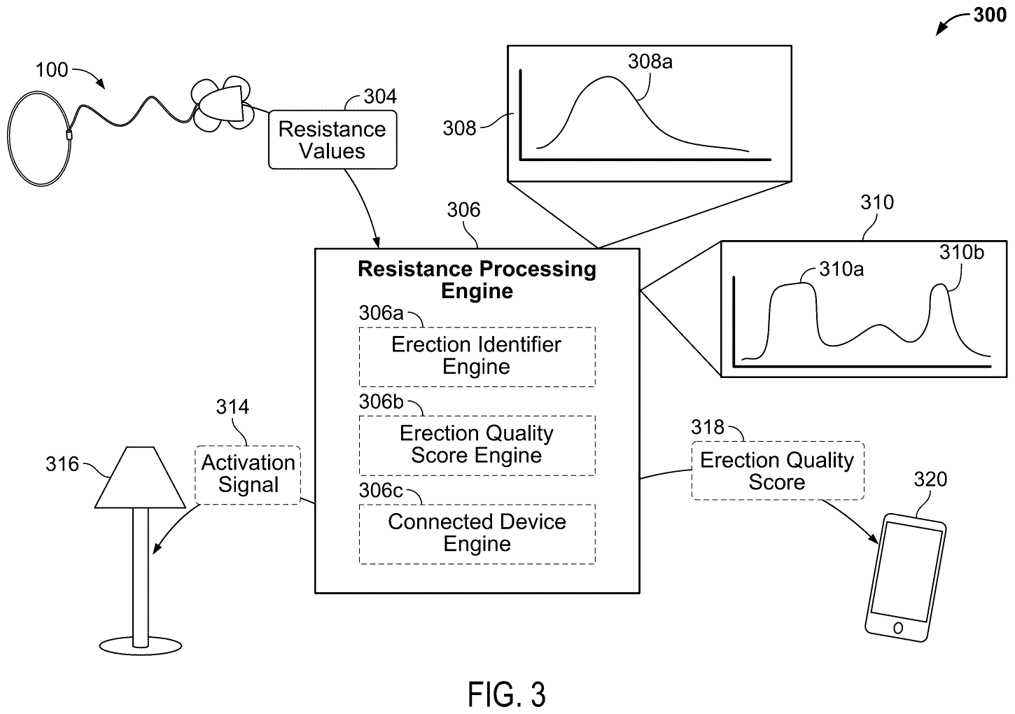

FIG. 3 illustrates an example system 300 for determining erectile function using the device 100 of FIG. 1. The system 300 includes a resistance processing engine 306 that receives data from the device 100 for determining erectile function. The resistance processing engine 306 can generate displacement values that measure how much the band 104 of the device 100 is displaced by a volume of the penis 105b.

In general, the system 300 obtains resistance values 304 from the device 100 and performs an action in response to processing the resistance values 304. In some examples, the system 300 is included in a housing of the housing component 102. In some examples, the system 300 may be communicably connected to one or more elements of the housing component 102, e.g., via a wired or wireless connection. The system 300 may be used with other example wearable devices, but for ease of reference, the system 300 will be described with reference to the device 100 of FIG. 1.

Resistance values may be correlated with penis circumference, and therefore displacement of the band 104. For example, as a penis grows in circumference, the band 104 of the device 100 stretches. The expansion of the band 104 of the device 100 increases the electrical resistance through the band 104 because the pathway through the ionic material of the band 104 increases in length (e.g., the band gets longer and thinner as it stretches). The resistance processing engine 306 can use a correlation of resistance values and penis circumferences to determine penis circumference based on obtained resistance values. The resistance processing engine 306 can use the resistance values 304 to generate displacement values—e.g., how much the band 104 of the device 100 is displaced by a volume of the penis 105b. The system 300 can measure how much a penis expands. The system 300 can compare values indicating expansion with values indicating maximal girth of the penis to generate an erection quality score.

In some cases, a table of values, including band resistance factors, maximal girths, associated quality scores, or other data can be used to generate an erection quality score. For example, a table of values can include one or more values associated with a quality score. The system 300 can generate an erection quality score based on a comparison of one or more detected values for a given penis and one or more values within the table of values. The one or more values within the table of values can be associated with a particular quality score. In some cases, the system 300 can generate an erection quality score that matches the quality score or is scaled based on a difference between the one or more detected values and the one or more table values. For example, if the one or more detected values and the one or more table values match, the system 300 can generate an erection quality score that matches the quality score. If the one or more detected values and the one or more table values do not match, the system 300 can generate an erection quality score that is scaled based on the quality score and a scaling function, e.g., linear scaling, exponential scaling, quadratic scaling, among others. An erection quality score can include an indication of rigidity of a penis.

The system 300 may be embodied in one or more circuit elements included in a housing component, such as the PCB 216a of the housing component 102. The system 300 may be embodied in one or more computers, e.g., one or more computers communicably connected to the device 100. One or more computers may include a personal electronic device, such as a smartphone or laptop, that is configured to receive signals indicating the resistance values 304 or a processed result of the system 300.

The resistance processing engine 306 obtains the resistance values 304 from the device 100. The resistance values 304 may indicate electrical resistance within a flow path of the band 104 of the device 100. The resistance values may be processed and graphically displayed.

For example, items 308 and 310 show example graphs of different individuals showing how resistance values may change over time during an erection session. In the graphs, the x-axis is time, and the y-axis can be an indication of penis circumference, such as a percentage of maximum known girth, e.g., where 100% corresponds to an erection that matches a maximum known girth. Graphs can be provided to a user. Graphs can include any appropriate measurements for the y-axis that indicate an erection, such as length measurements, resistance values, % of maximum girth, among others.

Item 308 is indicative of an older individual with severe erectile dysfunction. The graph of item 308 shows results of an individual who can achieve, but not maintain, an erection during an erection session. The device 100 records a high peak 308a corresponding to an erection that does not last very long, e.g., less than a minute. This data may be provided to a user, such as the individual, medical professional, or other, to improve diagnostic or treatment plans. Item 310 is indicative of a younger individual with psychogenic erectile dysfunction. The graph of item 310 shows results of an individual who can periodically achieve good erections (e.g., at 310a and 310b) during an erection session. Again, this data may be provided to a user, such as the individual, medical professional, or other, to improve diagnostic or treatment plans. Items 308 and 310 are example interpretations of data obtained via the device 100.

In some cases, the resistance processing engine 306 includes one or more engines, such as subengines. The one or more engines can perform one or more operations based on the resistance values 304. For example, an erection identifier engine 306a identifies one or more erections in one or more erection sessions. An erection quality score engine 306b can determine one or more quality scores of one or more erections. A connected device engine 306c can provide one or more data signals to connected devices, such as an activation signal 314 to a light 316 configured to turn on the light 316 or data indicating an erection quality score 318 provided to a personal electronic device 320.

In some cases, the resistance processing engine 306 determines a start of an erection session using the obtained resistance values 304. The resistance values 304 can include noise or other values that were not generated based on an erection, but were instead generated, for example, when a user first places the device 100 around the penis, adjusts a fit, or other action that affects resistance values. The resistance processing engine 306 can process the obtained resistance values 304 and determine whether a subset of values indicates an erection session or indicates noise or other non-erection actions, such as, for example, stretching the band while adjusting or putting on the device 100.

When the device 100 is worn by a user during sexual activity, the device 100 is configured to transmit, via a wired or a wireless connection, the resistance values 304 to the resistance processing engine 306. In some cases, the erection identifier engine 306a identifies a start of an erection session, e.g., a time period when a penis satisfies a threshold circumference or resistance value. For example, the erection identifier engine 306a can use a set of one or more resistance values to determine that an erection session started at a time corresponding to the one or more values. The erection identifier engine 306a can include various processing to determine a start of an erection session, such as resistance or time thresholds. For example, the erection identifier engine 306a can determine whether a given resistance value, or value range, is measured over a threshold amount of time. If the value, or value range, is maintained and satisfies a threshold time, the erection identifier engine 306a can identify a corresponding start of an erection session.

In some cases, in response to identifying an erection session, the erection identifier engine 306a can remove one or more resistance values from memory. For example, the erection identifier engine 306a can be connected to a memory device configured to store the resistance values 304. An erection session can be identified by a subset of resistance values. After identifying an erection session, e.g., at the start of an erection session or subsequently in post processing scenario, the erection identifier engine 306a can delete the subset of resistance values that are not indicative of the erection session. In this way, the erection identifier engine 306a can help reduce storage requirements, e.g., by removing from memory data that is not indicative of erection sessions.

In some cases, the erection identifier engine 306a determines a start of an erection session based on resistance values satisfying a threshold. For example, at least a subset of the resistance values 304 can indicate a penis circumference that satisfies a threshold circumference. The threshold circumference can be a static value for all users or can be user-specific. A threshold circumference can be provided using an input interface where a user provides a circumference or can be determined based on user data, e.g., a user specifying a time period where an erection occurred or a pre-measured baseline value.

In some cases, the erection identifier engine 306a includes one or more models that learn to identify an erection session based on obtaining ground truth data from a user indicating when an erection session occurred, e.g., a time range, start, or finish. The erection identifier engine 306a can provide resistance values as input data to one or more models. The erection identifier engine 306a can compare output of the models to one or more ground truth data items indicating one or more erection sessions. Based on error between the output of the models and the ground truth data, the erection identifier engine 306a can update the one or more models, e.g., updating one or more parameters or layers of one or more models.

In some cases, the erection quality score engine 306b determines an erection quality score of an erection session. The erection quality score engine 306b can estimate a current circumference of the penis measured by the device 100 and a previously stored circumference of the penis to generate a quality score. The stored circumference can indicate a maximum circumference of the user—e.g., measured using the device, calipers, fabric ruler, string, or other measurement tool by the user. The erection quality score engine 306b can determine one or more values of displacement, e.g., using the resistance values 304, where values of displacement indicate an amount that a volume of the penis 105b displaces the band 104 of the device 100. Using the one or more values of displacement, the erection quality score engine 306b can generate one or more erection quality scores.

The erection quality score engine 306b can compare a current circumference, indicated by the obtained resistance values 304, to the stored circumference to determine a personalized quality score for an erection. For example, a person with a small penis may have an erection with a circumference of X inches. The quality score of this erection compared to a larger penis with a maximum circumference of Y (where Y>>X) would be poor but, compared to the maximum circumference (e.g., a baseline value) of the smaller penis, the quality score may be higher. Quality scores can be represented in any suitable alphabetic, numeric, alphanumeric, color codes, or other indications. In some cases, a quality score includes a ratio of a current penis circumference over a maximum circumference (e.g., baseline value) for the same penis. A quality score can be measured throughout an erection session or calculated as an average, e.g., by comparing an average session circumference with a maximum circumference or by comparing a maximum session circumference with a previously stored maximum.

In some cases, the connected device engine 306c performs an action based on the processing of one or more resistance values. For example, the connected device engine 306c can provide an indication of the erection quality score to a user. The erection quality score 318 can be indicated via a transmitted signal configured to display the erection quality score 318 on a display of a device, such as the personal electronic device 320. A quality score can be a number or a set of numbers indicating a quality score at different points of time within an erection session or can indicate an average quality score for a session. The connected device engine 306c can transmit an indication of one or more quality scores to one or more devices in real-time—e.g., during a sexual activity that causes an erection session to be measured, the connected device engine 306c can provide indications to one or more devices. Indications can include values, graphs, charts, images, or other displays.

In some cases, providing an indication includes providing an indication during sexual activity. The system 300 can generate and provide output to a device, such as the personal electronic device 320 which can include a smartphone, laptop, or other device, information that can be useful to a wearer or a sexual partner of the wearer. Information can include updates on one or more of a current erection session, quality scores, motivational messages, information on goal achievement, suggestions for changing sexual positions or activities, etc. Information can be provided via audio, visual (e.g., display, lighting), or tactile (e.g., vibration of the device) feedback. Goals for erectile function can be set by a user before sexual activity begins, e.g., in consultation with a medical professional or during setup of an application that pairs with the device 100. The system 300 can be used to provide updates on these goals in real time, including providing specific feedback tailored to the user, e.g., motivational indications, critical indications, notes for sexual adjustments based on sensed data of a current sexual activity using one or more sensors, among others.

In some cases, multiple erection sessions can be combined for providing feedback, such as trending or historical data. For example, the system 300 can calculate averages or other metrics based on one or more sessions. The system can generate various indications, such as overlaid plots or comparisons of times for one or more sessions. Times can indicate a time it takes to achieve an erection for each session or a time an erection was maintained. Feedback can include a summary of one or more sessions. Summaries can be generated using one or more trained models, such as large language models. Feedback can include questions for a user, such as psychological questions to help gauge one or more psychological factors that could impact erectile performance. Feedback can include notifications to recalibrate a sensor, e.g., after a detected change in average size of a penis caused by growth or a change in user.

In some cases, the system 300 determines rigidity using one or more previously measured factors of girth, known resistive forces, and actively-monitored displacement. For example, the system 300 is configured to determine a rigidity of an erection by obtaining the resistance values 304. The system 300 can use the resistance values 304 to determine a penis circumference over time. The system 300 can use resistive forces, such as a known resistance of a band worn on the penis, to help determine rigidity, circumference values, or quality scores. In some cases, previous factors of girth, such as a maximum circumference, can be compared with current circumference measurements to determine a personalized indication of a quality of an erection. Resistive forces of a band 104 of the device 100 can further be used to improve accuracy of determined values, such as determined values of circumference or quality scores. In general, a circumference closer to a maximum, maintained for longer, and where a band has a higher resistive force, can indicate an erection that is more rigid or of higher quality than a circumference further from a maximum, maintained for a shorter time, and where a band has a lower resistive force.

In some cases, the connected device engine 306c activates one or more devices. For example, the connected device engine 306c can transmit the activation signal 314 to a light 316. The light 316 can communicate with an IoT device or a device with an element for receiving a signal from the system 300. The light 316 can be configured to activate a light in response to receiving the activation signal 314. The connected device engine 306c can generate the activation signal 314 to cause the light 316 to change colors, change intensity, switch on or off, or a combination of these among others.

In some cases, the resistance processing engine 306 compares one or more generated quality scores to one or more stored erection quality scores. For example, the resistance processing engine 306 can compare data, such as erection quality scores, of a user with the same user or other users. For quality scores, comparison with other users can indicate whether a current user is having higher or lower quality erection scores compared to a population, such as other users of a device or other pools of data. Comparison data can include local, on device data, or cloud data.

In some cases, the resistance processing engine 306 determines a series of two or more circumference measurements of a penis. For example, during one or more erection sessions, the resistance processing engine 306 can determine two or more circumferences based on one or more electrical resistance values. In some cases, the resistance processing engine 306 determines a measure of rigidity using two or more determined circumferences. For example, the resistance processing engine 306 can calculate an average rigidity based on two or more determined circumferences over time. Rigidity can be equal to area under a curve formed by circumference measurements over time.

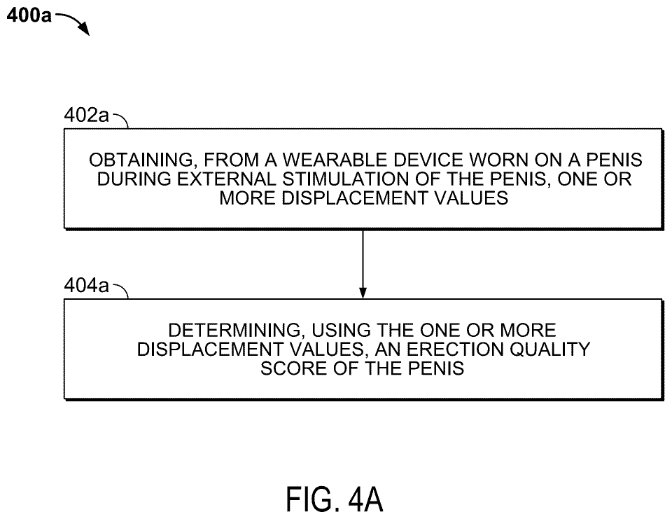

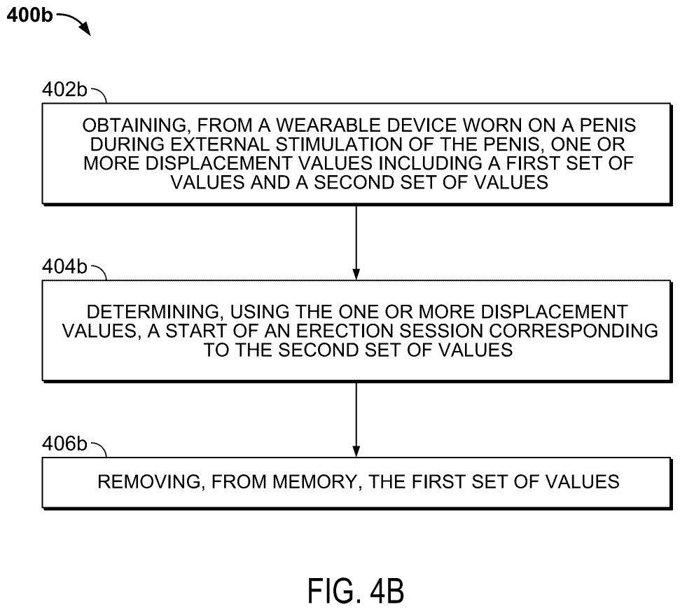

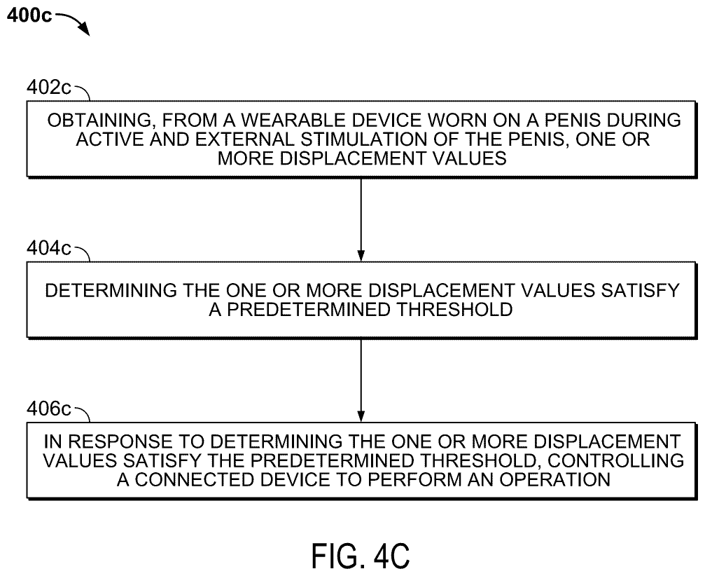

FIG. 4A is a flowchart of an example process 400a for determining an erection quality score. For convenience, the process 400a will be described as being performed by a system of one or more computers, located in one or more locations, and programmed appropriately in accordance with this specification. For example, the system 300 of FIG. 3, appropriately programmed, can perform the process 400a using resistance values captured using device 100 of FIG. 1 as displacement values to determine an erection quality score.

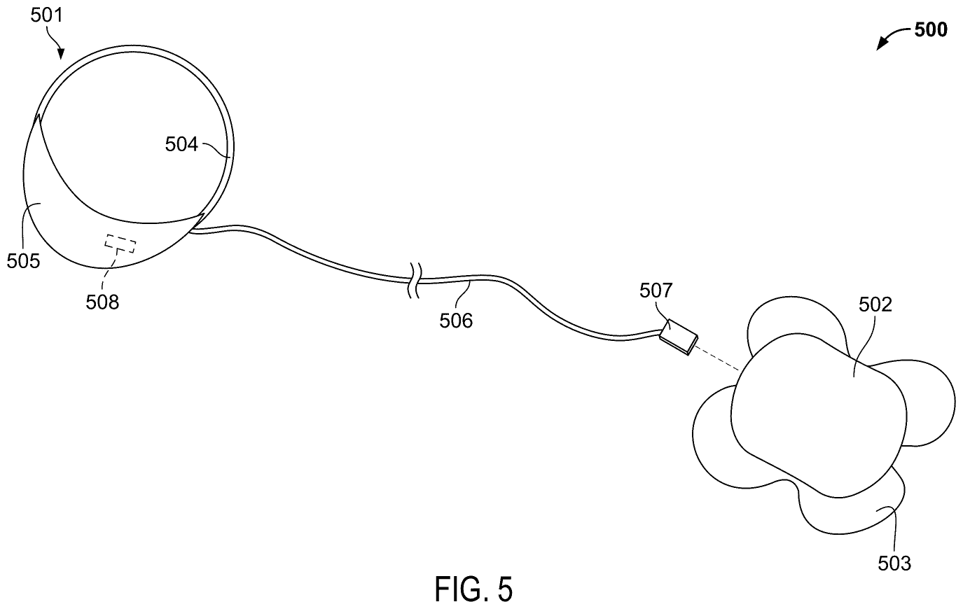

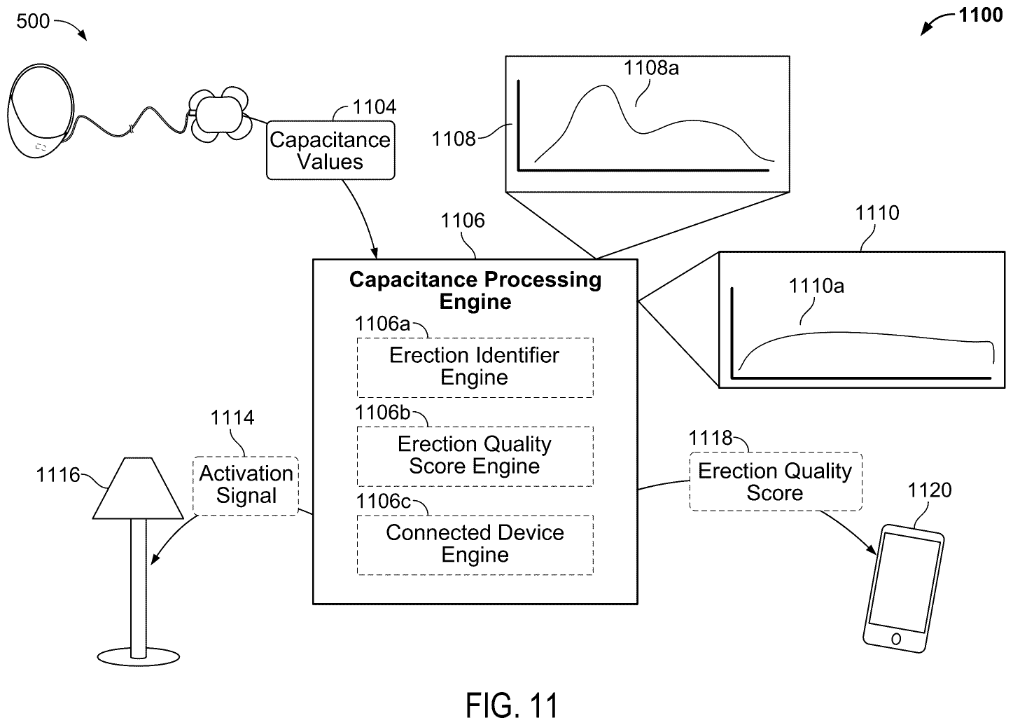

While the device 100 of FIG. 1 and system 300 of FIG. 3 are described above, the process 400a can be performed by a variety of systems using a variety of wearable devices configured to capture displacement values. As another example, the system 1100 of FIG. 11, appropriately programmed, can perform the process 400a using capacitance values captured using device 500 of FIG. 5 as displacement values to determine an erection quality score. For more details on device 500 or system 1100, refer to FIGS. 5 and 11, respectively.

Initially, the process 400a may include a calibration step in which a max circumference of an individual's penis is measured and stored as, or associated with, a baseline value. For example, the individual's penis can be associated with a resistance value or a capacitance value.

The process 400a includes obtaining, from a wearable device worn on a penis during stimulation of the penis, one or more displacement values (402a). For example, the system 300 can obtain resistance values 304 from the device 100 as displacement values when the device 100 is worn on a penis during sexual activity. As another example, the system 1100 can obtain capacitance values 1104 from the device 500 when the device 100 is worn on a penis during sexual activity.

The process 400a includes determining an erection quality score of the penis (404a) using the one or more displacement values. For example, the system 300 is configured to determine the erection quality score 318 using the resistance values 304 and a previously stored value indicating a first circumference of the penis (e.g., the baseline or max circumference value). As another example, the system 1100 is configured to determine the erection quality score 1118 using the capacitance values 1104. An erection quality score can help indicate erectile function for a given user.

In some cases, the process 400a includes performing a stress test on the penis. For example, an individual wearing the wearable device can be instructed to engage in an erection session over a period of time, e.g., ten minutes. Data obtained by the wearable device during this erection session can be compared with known values, such as values indicating a circumference of the penis. Through such a comparison, the wearable device, or element communicably connected to the wearable device, can generate an erection quality score. A person who is young and healthy may be able to maintain a fully rigid erection for ten minutes or more. Someone who has erectile dysfunction may only achieve a partial erection during that time or lose it quickly. In some cases, area under a curve, such as a displacement curve, can be used to generate the erection quality score. For example, the system 300 or the system 1100 can include an element that generates a curve indicating erection circumference and can further generate the erection quality score based on the curve.

In some cases, the process 400a includes providing an indication of the erection quality score to a user. For example, the system 300 can provide the erection quality score 318 to the personal electronic device 320. As another example, the system 1100 can provide the erection quality score 1118 to the device 1120. An indication of an erection quality score can include graphs, images, video, values, among others. Providing the indication can include transmitting the indication of the erection quality score to a display. For example, the system 300 or 1100 can transmit an indication of the erection quality score 318 or 1118 to a display of the personal electronic device 320 or 1120, e.g., where the transmission is configured to cause a display of the personal electronic device 320 or 1120 to display an indication of the erection quality score 318 or 1118. An indication of an erection quality score can be transmitted to one or more devices in real-time, such as via a broadcasted signal. The indication can include an indication of whether an erection is of high or low quality, time-based analysis of an erection session, among other elements.

In some cases, the process 400a includes comparing the erection quality score to one or more erection quality scores. For example, the system 300 or the system 1100 can compare the erection quality score 318 or 1118 to one or more other quality scores of a same wearer of a device or other person. The comparison can provide information as to a quality of erection or ways to improve erectile function (e.g., by performing similar actions or treatment as similar other users that have better scores or have improved scores over time).

In some cases, the process 400a includes determining, using the one or more displacement values, a series of two or more circumference measurements of the penis. For example, the system 300 can determine two or more circumference measurements of a penis measured by the device 100 using the resistance values 304. As another example, the system 1100 can determine two or more circumference measurements of a penis measured by the device 500 using the capacitance values 1104. For example, the resistance values shown in items 308 and 310, and the capacitance values shown in 1108 and 1110 can be used to generate one or more series of two or more circumference measurements of at least one penis.

In some cases, the process 400a includes calculating an average rigidity of the penis using the series of two or more circumference measurements over time. For example, the system 300 can calculate an average rigidity of the penis measured by the device 100, or the system 1100 can calculate an average rigidity of the penis measured by the device 500. The average rigidity can include determining an area under a curve formed by determining a circumference of a penis over time, where an erection session can include a penis becoming more or less erect as a function of time and where maintaining a hard, more constant, erection corresponds to a higher quality erection compared to maintaining a less hard, less constant erection. Average rigidity can be higher if a penis is determined to be at a larger circumference for longer compared to a penis being at a smaller circumference in less time.

In some cases, determining the erection quality score of the penis includes calculating a rigidity of the penis using the one or more resistance values and the stored value indicating the first circumference of the penis. For example, resistance values can be used to determine an indication of penis circumference. As another example, capacitance values can be used to determine an indication of penis circumference. This indication can be used with the first circumference to determine an erection quality score or calculate a rigidity. For example, the system 300 can use one or more algorithmic processes of trained models to process input indicating the resistance values 304 and a stored circumference of the penis (such as a maximum circumference achieved by the penis) to determine the erection quality score or calculate a rigidity. As another example, the system 1100 can use one or more algorithmic processes of trained models to process input indicating the capacitance values 1104 to determine the erection quality score or calculate a rigidity.

In some cases, the process 400a includes determining a second circumference of the penis using the one or more displacement values; and determining, using the first circumference and the second circumference, the erection quality score. For example, a stored maximum can be used to determine a ratio of maximum to current circumference, where high quality erections can include erections where the measured circumference is within a threshold from a maximum for a threshold period of time.

In some cases, the process 400a is performed using a device that includes a band that is configured to encircle the penis, and the process 400a includes obtaining a resistance factor of the band; and determining the erection quality score of the penis using the resistance factor. For example, the resistance factor of the band can represent an elasticity of a material used to form the band. The resistance factor can refer to mechanical resistance of the band to expansion where greater force is required to displace a band with a resistance factor that indicates greater resistance. As an example, the band can be used to obtain electrical resistance values, such as the resistance values 304. As another example, the band can be used to obtain capacitance values 1104. In some cases, a resistance factor of the band can help determine a quality of an erection because each segment of displacement of a more mechanically resistant band indicates greater erection strength compared to a less mechanically resistant band.

In some cases, determining the erection quality score uses (i) the resistance factor and (ii) a surface area of the band. For example, the system 300 or the system 1100 can use a mechanical resistance factor of a band 104 of the device 100 or a band 504 and a surface area of the band 504 of the device 500 to determine an erection quality score. In some cases, the resistance factor includes an indication of a surface area of the band, such as a measure of pressure in Pascals or a measure of force in Newtons. In general, a greater surface area and a greater resistance factor can require greater force by the penis to displace the band. Greater erectile force can indicate a better-quality erection. In some cases, a resistance factor of the band can be minimal enough not to impede a user from achieving an erection, e.g., by overly restricting blood flow to the penis.

In some cases, erectile rigidity and quality scores can be generated using values indicating a resistance factor of the band. The system 300 or 1100 can account for a known extent of resistance of the device 100 or 500 at various stretched dimensions. For example, when the band 104 or 504 is in a fully contracted state, there would be no or little resistance from the band on the penis 105b, e.g., when the penis 105b is smaller or equal to a circumference of the band 104 or 504 in a fully contracted state or when the band 104 or 504 is not being worn on the penis 105b. When the band 104 or 504 is in a fully extended state, it may exert approximately 5 Newtons of force over the surface of the band 104 or 504, e.g., where the band 104 or 504 is configured to provide a pressure of 5 Newtons divided by the surface of the band 104 or 504. The system 300 or 1100 can calculate an extent of displacement using values indicating a maximum erection. The system 300 or 1100 can use the extent of displacement to determine a quality score, e.g., including or indicating an erectile rigidity. A quality score can be determined, in some cases, by looking up one or more values in a table of values—e.g., looking up values indicating a resistance factor of the band 104 or 504, a maximum erection for a given person, or current displacement values of an erection.

In some cases, a device used for the process 400a includes a tube 101 that encloses an ionic material, the tube 101 being configured to encircle the penis; and where obtaining the one or more resistance values includes obtaining one or more resistance values representing a resistance of an electrical current flowing through the ionic material of the tube 101. For example, the system 300 can obtain the resistance values 304 from the device 100 described above. The device 100 can include a tube that encloses an ionic material and that is configured to encircle the penis. Ionic material can include ions within a substance where the ions help to transfer an electric current via electrons through the tube from a negative charge end to a positive charge end. The resistance values 304 can indicate a resistance of an electric current through the ionic material of the tube of the device 100.

In some cases, a device used for the process 400a includes a resistance sensing circuit, where obtaining the one or more resistance values includes obtaining one or more resistance values representing a resistance measured by the resistance sensing circuit. For example, the wearable device 100 can include a resistance sensing circuit, e.g., included on a circuit board, such as the circuit board 216a of FIG. 2. The sensing circuit can be configured to generate the resistance values 304, e.g., based on sensing elements of an electric current flowing through a band portion of the device 100.

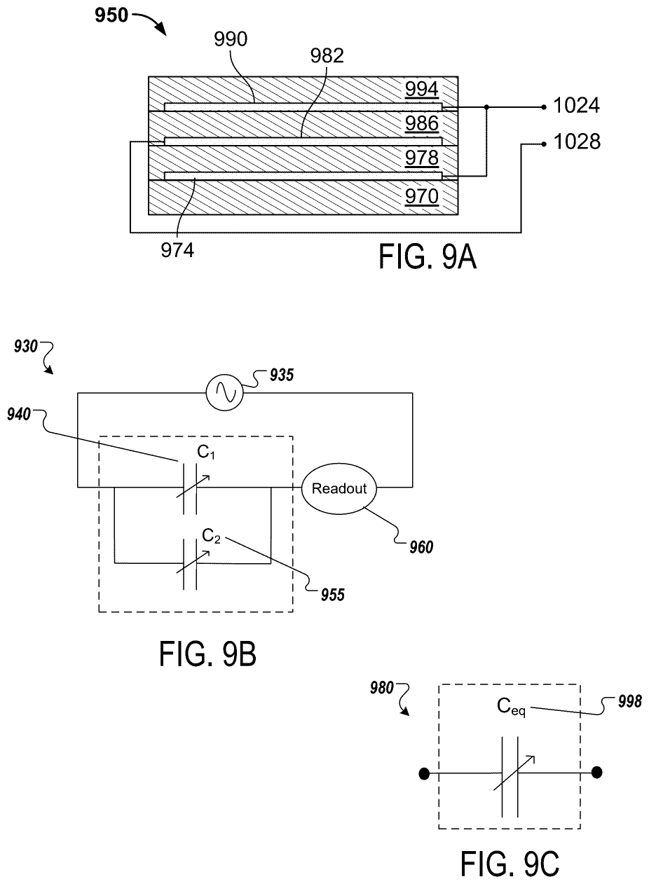

In other cases, a device used for the process 400a includes an encapsulated layered assembly. The encapsulated layered assembly incudes a first layer of conductive material, a second layer of conductive material arranged approximately in parallel with the first layer of conductive material, and a dielectric layer disposed between the first and second layers of conductive material. The layered assembly may have one or more additional conductive material layers or one or more additional dielectric layers. The encapsulated layered assembly is included in a band with an interior surface placed against the penis of the user, and an exterior surface opposite the interior surface. The band has a base layer that defines the interior surface of the body, and a top layer that defines the exterior surface of the body. The device can capture a capacitance value that specifies an ability to store electric charge between at least the first layer of conductive material and the second layer of conductive material, e.g., as is described in more detail with respect to FIGS. 9A-9C.

In some cases, the process 400a includes activating a device by generating an activation signal using another device; and providing the activation signal to the device. For example, the system 300 or 1100 can include a device, such as the personal electronic device 320 or 1120. The personal electronic device 320 or 1120 can generate an activation signal that activates another device, such as the wearable device 100 or the wearable device 500. For example, a user interface on the personal electronic device 320 or 1120 can allow a user to make a selection that causes the personal electronic device 320 or 1120 to send a signal to activate the wearable device 100 or 500. Activating the wearable device 100 or 500 can include turning on the wearable device 100 or 500, e.g., to start sensing or generating current for determining erectile function.

In some cases, providing the activation signal to the wearable device includes sending a short-range wireless communication to the wearable device. For example, the system 300 or 1100 can provide an activation signal to the device 100 or 500 via a wireless communication, such as a short-range wireless communication. Examples of wireless communication can include Wi-Fi, Bluetooth, near field communication, or a combination of these among others.

FIG. 4B is a flowchart of an example process 400b for determining a start of an erection session using displacement values. Again, for convenience, the process 400b will be described as being performed by a system of one or more computers, located in one or more locations, and programmed appropriately in accordance with this specification. For example, the system 300 of FIG. 3, appropriately programmed, can perform the process 400b using one or more resistance values as displacement values.

While the system 300 of FIG. 3 is described above, the process 400b can be performed by a variety of systems. As another example, the system 1100 of FIG. 11, appropriately programmed, can perform the process 400b using capacitance values as displacement values. For more details on system 1100, refer to FIG. 11.

Initially, the process 400b may include a calibration step in which a max circumference of an individual's penis is measured and stored as the baseline value. The process 400b includes obtaining, from a wearable device worn on a penis during sexual activity, one or more capacitance values including a first set of values and a second set of values (402b). For example, the system 300 can obtain the resistance values 304. As another example, the system 1100 can obtain the capacitance values 1104. The resistance values 304 or the capacitance values 1104 can include a first and second set of values. The first set of values can indicate noise values, e.g., from a user first putting on the device, adjusting a fit, or otherwise causing values not indicating an erection. The second set of values can include values indicating an erection.

The process 400b includes determining, using the one or more capacitance values, a start of an erection session corresponding to the second set of values (404b). For example, the system 300 can identify an erection using one or more values of the resistance values 304. As another example, the system 1100 can identify an erection using one or more values of the capacitance values 1104. An erection session can correspond to values indicating a relative value range for a threshold period of time, e.g., relative to a maximum stored circumference. In some cases, a fixed threshold value set at or associated with the maximum circumference is used to identify non-erection values. As an example, resistance values 304 or capacitance values 1104 indicating a circumference larger than a maximum known circumference must be erroneous or indicative of non-erection values unless measured for more than a threshold period of time, in which case a notification to reconfigure the device can be provided to a user. In some cases, dynamic thresholds are used.

In some cases, trained models are used. For example, a model can be trained to detect, based on a set of one or more displacement values, the start or end of an erection session. The model can be trained using training data indicating known erection sessions, or sessions in among non-erection data. Training can include one or more models predicting a start, end, or time range of an erection session and comparing the prediction to a ground truth value. One or more models can be adjusted based on the comparison to help improve the accuracy of the models in correctly detecting a start of an erection. Models can be run on a device of the system 300 or 1100, e.g., a connected smartphone or server.

The process 400b includes removing, from memory, the first set of displacement values (406b). For example, the system 300 can store one or more of the resistance values 304 in memory, such as random-access memory (RAM) or other forms of computer memory. As another example, the system 1100 can store one or more of the capacitance values 1104 in memory, such RAM or other forms of computer memory. The displacement values can be stored for processing. In response to determining that a portion of the resistance values 304 or the capacitance values 1104 do not indicate an erection—e.g., rather indicating noise or other non-erection activity—the system 300 or the system 1100 can remove the values. In some cases, removing values that are not indicative of erections can reduce storage requirements of the system 300 or the system 1100. Data from one or more erections can be moved to long term storage either on a cloud storage system or on memory allocated within one or more devices included in the system 300 or the system 1100.

In some cases, determining the start of the erection session corresponding to the second set of values includes determining the second set of values indicate a penis circumference that satisfies a predetermined threshold. For example, the system 300 or 1100 can determine that a first set of values indicates a flaccid penis in response to determining that the first set indicates or is associated with a circumference that satisfies a flaccid circumference value. The system 300 or 1100 can determine a second set of values indicates an erect penis in response to determining that the second set indicates or is associated with a circumference that satisfies an erection circumference value. Values can be static, e.g., based on or in proportion to a maximum known circumference of a penis, or may be dynamic—e.g., adjusted based on training one or more models or based on historical erection sessions recorded by the system 300 or the system 1100 and later used for retraining or refining the system 300 or 1100.

FIG. 4C is a flowchart of an example process 400c for controlling connected devices in response to determining erectile function. Once more, for convenience, the process 400c will be described as being performed by a system of one or more computers, located in one or more locations, and programmed appropriately in accordance with this specification. For example, the system 300 of FIG. 3, appropriately programmed, can perform the process 400c.

While the system 300 of FIG. 3 is described above, the process 400c can be performed by a variety of systems. As another example, the system 1100 of FIG. 11, appropriately programmed, can perform the process 400c. For more details on system 1100, refer to FIG. 11.

Initially, the process 400c may include a calibration step in which a max circumference of an individual's penis is measured and stored as the baseline value. The process 400c includes obtaining one or more displacement values from a wearable device worn on a penis during sexual activity. For example, the system 300 can obtain the resistance values 304 from the device 100. As another example, the system 1100 can obtain the displacement values 1104 from the device 500.

The process 400c includes determining the one or more resistance values satisfy a predetermined threshold (404c). For example, the system 300 can determine that one or more of the resistance values 304 satisfy one or more thresholds. As another example, the system 1100 can determine that one or more of the capacitance values 1104 satisfy one or more thresholds. One or more thresholds can help indicate a quality of an erection, rigidity of an erection, erection session length, erectile function goals, or a combination of these among others. In some cases, determining the values satisfy a threshold can include processing the resistance values or capacitance values to generate or identify associated circumference values, and comparing the processed values to thresholds. In other cases, determining the values satisfy a threshold can include directly comparing resistance values or capacitance values to thresholds.

The process 400c includes in response to determining the one or more resistance values satisfy the predetermined threshold, controlling a connected device to perform an operation (406c). For example, the system 300 includes the connected device engine 306c, and the system 1100 includes the connected engine 1106c. In general, engines can be configured using software or hardware elements. The connected device engine 306c or 1106c can connect to one or more devices to perform an operation, such as displaying indications of an erection session or activating systems to change lighting, make sound, vibrate the housing component of the device 100 or 500, or cause other actions to occur.

In some cases, controlling the connected device to perform the operation include controlling a light to change an illumination state. For example, the system 300 can provide the activation signal 314 to the light 316, where the activation signal 314 is configured to change an illumination state of the light 316, e.g., from on to off, off to on, from one color to another color, from one intensity to another intensity, among other changes. As another example, the system 1100 can provide the activation signal 1114 to the light 1116, where the activation signal 1114 is configured to change an illumination state of the light 1116, e.g., from on to off, off to on, from one color to another color, from one intensity to another intensity, among other changes.

In some cases, controlling the light to change the illumination state includes controlling the light to change color. For example, the system 300 or the system 1100 can provide the activation signal 314 or 1114 to the light 316 or 1116 where the signal is configured to change a color of the light 316 or 1116 from a first color to a second color, e.g., from red to green.

In some cases, controlling the light to change the illumination state includes controlling the light to change intensity. For example, the activation signal 314 or 1114 can be configured to increase an intensity of light. Changes to illumination can be made over time to effectively indicate information to a wearer of the device 100, device 500, or other person. Changes to illumination can be used to indicate that a goal has been reached or other detected aspect of monitored sexual activity.

In some cases, controlling the connected device to perform the operation includes controlling a speaker to emit audio. For example, the system 300 or 1100 can provide a signal to a device configured to make noise. The noise can indicate information of a monitored sexual activity, such as a quality of an erection or an achieved goal. The noise can be spoken audio, music, tones, or other sound. Specific noise can be associated with specific information such that the noise can effectively indicate information to a person.

In some cases, controlling a speaker to emit audio includes controlling the speaker to indicate a quality of erection based on the one or more displacement values, e.g., resistance or capacitance values. For example, the speaker may use various noises, including tones, music, spoken words and/or numbers, or a combination of one or more of these among others, to indicate aspects of monitored sexual activity, such as quality or duration of the erection.

In some cases, controlling the connected device to perform the operation includes controlling a vibration mechanism coupled to the band or the housing component of the device to emit vibrations. For example, the system 300 or the system 1100 can provide a signal to the vibration mechanism of the device 100 or 500. The vibration can indicate information of a monitored sexual activity, such as a quality of an erection or an achieved goal. The vibration can be short or long pulses, pulses with short or long gaps between each pulse, or vibrations with varying intensity or vibration patterns. Specific vibrations can be associated with specific information such that the vibration can effectively indicate information to a person.

Various forms of feedback, such as light adjustments or audio or tactile feedback, can help improve feedback during sexual activity by eliminating the need for a wearer or other person to check a display or engage in other behavior that could detract from a sexual activity.

While the band 104 of the device 100 of FIGS. 1 and 2 comprises an ionic fluid to sense displacement of a user's penis, in other examples, a band of a wearable device for determining erection quality may have a layered construction for sensing displacement, as shown in FIGS. 5-10. The device 500 for determining erectile function can be used with any of the methods of FIGS. 4A-4C. Elements of the device 500 in FIGS. 5-10 that are similar to the elements of the device 100 are designated by the same reference numeral, incremented by 400. A description of many of these elements is abbreviated or even eliminated in the interest of brevity.

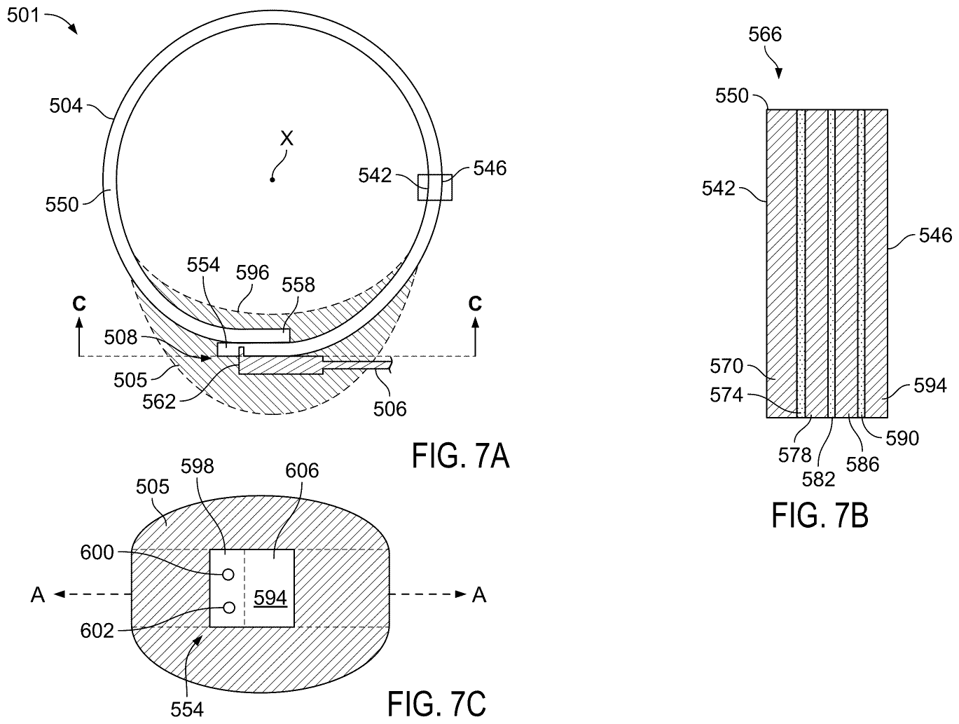

The wearable device 500 has a sensor assembly 501, a housing assembly 502, and electrical connection 506 coupling the sensor assembly 501 to the housing assembly 502. A first end (see FIG. 7A, first end 562) of the electrical connection 506 is coupled to the sensor assembly 501 at a juncture 508. The sensor assembly 501 includes a band 504 configured to be worn around a penis of a user of the wearable device and a setting portion 505 surrounding (e.g., molded or formed over) the juncture 508. The electrical connection 506, which includes two or more electrical leads, extends from the setting portion 505 and terminates at connector 507.

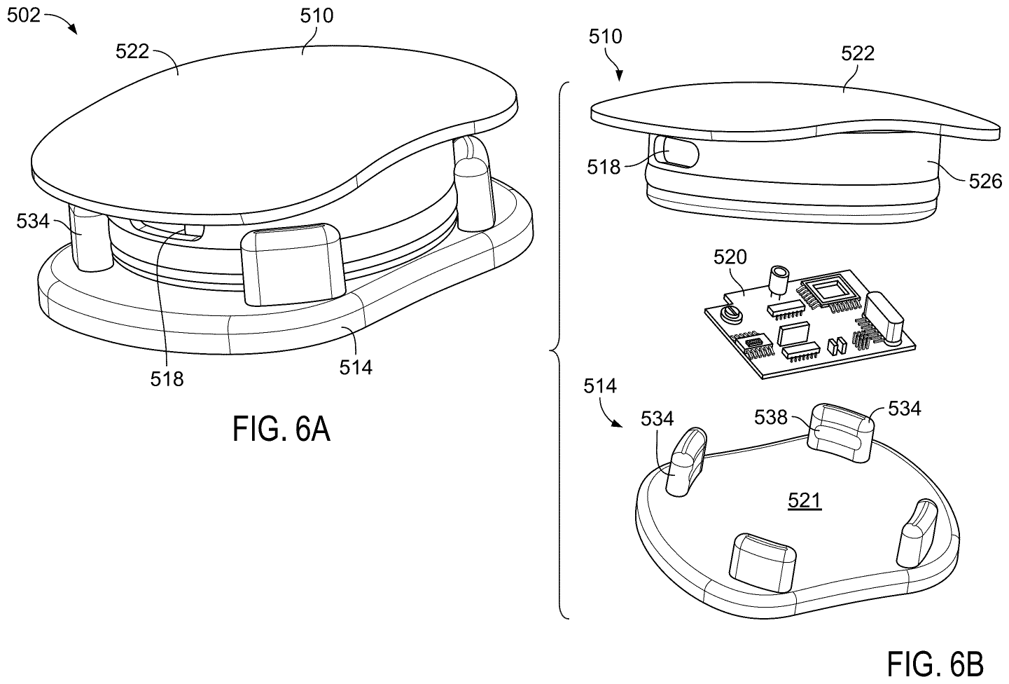

Turning to FIGS. 6A and 6B, the housing assembly 502 includes a cover 510 and a base 514. The base 514 is coupled to the adhesive 503 and attaches to the body of the individual. The cover 510 defines an opening 518 (e.g., electrical port) sized to receive the connector 507 of the electrical connection 506 and is removably attached to the base 514. A printed circuit board (PCB) 520 is disposed between the cover 510 and the base 514 of the housing assembly 502. The cover 510 and another barrier or cap may enclose the PCB 520 so that the PCB 520 is secured to the cover 510 so that the PCB 520 and the cover 510 are removably attached together from the base 514. The cover 510 has a curved cap 522 and a collar 526 extending from the cap 522. The collar 526 defines the opening 518 of the housing assembly 502 and has an annular ridge 530 extending continuously around a circumference of the collar 526. The ridge 530 is configured to mate with a plurality of posts 534 disposed at each rounded corner of the base 514. Each post 534 has a groove 538 sized to receive the ridge 530 of the cover 510. So configured, the cover 510 is rotatable relative to the base 514 while the housing assembly 502 remains securely assembled.

The opening 518 can be an electrical port that is configured to electrically connect to the connector 507 of the sensor assembly 501 (e.g., the electrical connection 506) for operating the wearable device 500. The electrical port 518 may also be configured to electrically couple to a power source for charging a battery disposed inside the housing assembly 502 (e.g., a battery connected to the PCB 520) or electrically couple the PCB 520 to an external processor, etc. While the opening 518 is oval-shaped in FIGS. 5-6B, in other examples, the opening 518 of the housing assembly 502 may be any suitable shape to receive a corresponding connector 507, charging cable, or other electrical wiring such as, for example, flexible printed circuit (FPC) connectors. In some examples, the housing assembly 502 may include one or more additional openings to receive one or more additional electrical connections.

The housing assembly 502 of FIGS. 6A and 6B may be easily disassembled by pulling the cap 522 away from the base 514. The base 514 and adhesive 503 may be disposable, while the cover 510 and the PCB 520 are reusable and may be removably attached to another base 514 for another erection session. However, in other examples, the housing assembly 502 may be disassembled by removing fasteners fixing the cover 510 to the base 514. In yet other examples, the cover 510 may not be rotatable relative to the base 514.

Referring now to FIG. 7A, the band 504 includes an interior surface 542 configured to be placed against the penis of the user, an exterior surface 546 opposite the interior surface, and an encapsulated, layered body 550. The body 550 has a first end 554 overlapping with a second end 558 to form a ring, such that the interior surface 542 of the first end 554 bonds with the exterior surface 546 of the second end 558. At juncture 508, the first end 554 of the body 550 is electrically coupled to the first end 562 of the electrical connection 506. The first end 562 of the electrical connection 506 and the first and second ends 554, 558 of the band body 550 are embedded in the setting portion 505 of the band 504. The electrical connection 506 extends through a side the setting portion 505 to couple to the housing assembly 502.

In FIG. 7B, an example layered assembly 566 of the body 550 of the band 504. includes a base layer 570, a first layer of conductive material 574 (e.g., conductive ink), a first layer of dielectric material 578, a second layer of conductive material 584, a second layer of dielectric material 586, a third layer of conductive material 590, and a top layer 594. The base layer 570 and top layer 594 define the interior and exterior surfaces 542, 546 of the body 550, respectively.

In FIG. 7A, the setting portion 505 is an elastic material that surrounds the first and second ends 554, 558 of the body 550 to integrate the juncture 508 with the band 504. A portion of the top layer 594 of the layered assembly 566, as shown exposed in the cross-sectional view of FIG. 7C, is adjacent to the first end 562 of the electrical connection 506. Returning to FIG. 7A, the setting portion 505 surrounds (e.g., by molding, printing, fusing, etc.) the overlapping first and second ends 554, 558 of the body 550 provide a smooth, and uninterrupted interior surface 596 that is flush (or substantially flush) with the interior surface 542 of the band 504. The setting portion 505 extends radially relative to axis X axis of band 504 to form a moon-shape, and as shown in FIG. 7C, the setting extends both laterally and longitudinally relative to a longitudinal axis A of the band 504 to form an oblong shape. So configured, the setting portion 505 provides a protective barrier to the electrical connection at the juncture 508, a smooth, continuous cover to the overlapping ends 554, 558 of the body 550, and a stabilizing shape with a non-slip exterior to keep the band 504 in place on the user during use.

In FIG. 7C, a first portion 598 of the first end 554 of the body 550 is configured to couple with the first end 562 of the electrical connection 506 at piercings 600, 602. As described further below, the first portion 598 of the first end 554 has an overall durometer that is greater than an overall durometer of a second portion 606 (e.g., the remaining portion of the body 550) of the body 550.

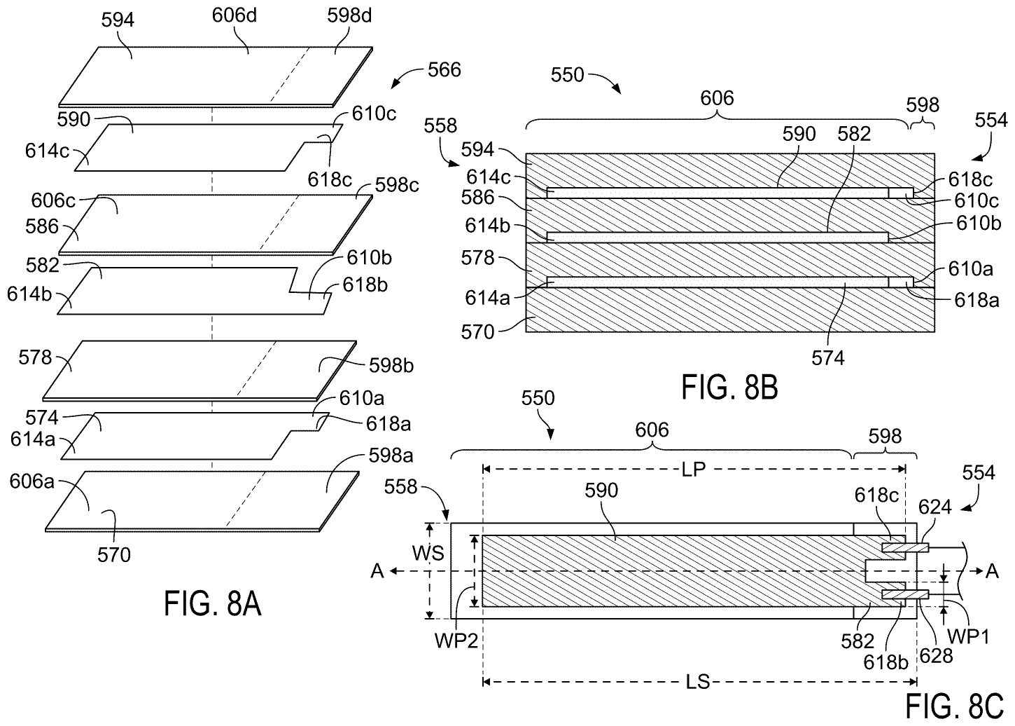

A method of fabricating a sensor assembly, such as the sensor assembly 501 of FIGS. 5-7C, is described with reference to FIGS. 8A-8C. While FIGS. 8A-8C refer to the components of the sensor assembly 501 previously described, the following method may be used or adapted to fabricate other layered sensors for measuring erectile function. The method includes applying the first layer of conductive material 574 on the base layer 570. The first layer of conductive material 574 may be applied by painting or printing conductive ink directly onto a layer of silicone material forming the base layer 570. The first layer of conductive material 574 has a first end 610a and a second end 614a opposite the first end 610a. The first end 610a terminates in a portion 618a configured to connect with an electrical lead of the electrical connection 506. The portion 618a is applied to the first portion 598a of the base layer 570.

The method includes applying the first layer of dielectric material 578 on the first layer of conductive material 574 such that a first end 598b of the first dielectric layer 578 covers the portion 618a of the first layer of conductive material 574. As shown in FIG. 8B, the first layer of dielectric material 578 extends beyond the ends 614a, 610a of the layer of conductive material 574. Returning to FIG. 8A, the second layer of conductive material 582 is then applied on the first layer of dielectric material 578 such that the second layer of conductive material 582 is parallel (or substantially parallel) to the first layer of conductive material 574. The second layer of conductive material 582 includes a portion 618b that aligns with the first portion 598b of the first dielectric layer 578 and is configured to connect with a different electrical lead of the electrical connection 506. The portion 618b of the second layer of conductive material 582 is offset relative to the portion 618a of the first layer of conductive material 574 about the longitudinal axis A of the body 550 (FIG. 8C). In some examples, the top layer 594 may be applied on the second layer of conductive material 582 to encapsulate a layered assembly 566. However, in the illustrated example of FIGS. 8A and 8B, the second layer of dielectric material 586 is applied on the second layer of conductive material 582 so that the first end 598c aligns with the portion 618b of the second layer of conductive material 582. The third layer of conductive material 590 is applied to the second layer of dielectric material 586 such that the third layer of conductive material 590 is parallel (or substantially parallel) with the first and second layers of conductive material 574, 582. As shown in FIG. 8A, a portion 618c of a first end 610c of the third layer of conductive material 590 is aligned with the first end 598c of the second dielectric layer 586 and with the portion 618a of the first layer of conductive material 574. The portion 618c is offset relative to the portion 618b of the second layer of conductive material 582. Finally, the top layer 594 is applied to the third layer of conductive material 590 to form an encapsulated body 550, as shown in FIG. 8B. A distal end 598d of the aligns with the portion 618c of the third layer of conductive material 590.

Turning now to FIG. 8C, the body 550 is coupled to the electrical connection 506 by piercing the first portion 598 of the first end 554 of the body 550 (at piercings 600, 602 of FIG. 7C) with first and second electrical leads 624, 628 of the electrical connection 506. The first electrical lead 624 pierces through the portions 618a, 618c of the first and third layers of conductive material 574, 590, and the second electrical lead 628 pierces through the portion 618b of the second layer of conductive material 582. The body 550 is shaped into a ring by bonding the exterior surface 546 of the second end 558 to the interior surface 542 of the second end, as shown in FIG. 7A. Finally, the setting portion 505 is coupled to the overlapping ends 554, 558 and the first end 562 of the first and second electrical leads 624, 628.

The method may include other preceding or intervening steps in fabricating the layered assembly 566. For example, applying any of the dielectric and top layers 578, 586, and 594 to the layers of conductive material 574, 582, 590 may include multiple steps of applying material. While the layered assembly 566 of FIGS. 7B and 8A-8C includes three layers of conductive material 574, 582, 590 and two layers of dielectric material 578, 586 separating the three layers of conductive material 574, 582, 590, in other examples, the layered assembly may have two layers of conductive material and one layer of dielectric material between the two layers of conductive material. In yet other examples, the layered assembly may have more than three layers of conductive material, with layers of dielectric material disposed between each layer of conductive material.

As shown in FIG. 8C, the portion 618 of the first end 610 of each layer of conductive material 574, 582, 590 has a width WP1 (e.g., 1.5 mm) in a range, for example, from about 0.5 mm or more (e.g., about 0.75 mm or more, about 1.0 mm or more, about 1.25 mm or more) to about 2.5 mm or less (e.g., about 2.25 mm or less, about 2.0 mm or less, about 1.75 mm or less, about 1.5 mm or less). The second end 614 of each layer of conductive material 574, 582, 590 has a width WP2 (e.g. 4.4 mm) in a range, for example, from about 3.0 mm or more (e.g., about 3.25 mm or more, about 3.5 mm or more, about 3.75 mm or more, about 4.0 mm or more, about 4.25 mm or more) to about 6.0 mm or less (e.g., about 5.75 mm or less, about 5.5 mm or less, about 5.25 mm or less, about 5.0 mm or less, about 4.75 mm or less, about 4.5 mm or less). A ratio of width WP1 to WP2 (WP1:WP2) may be in a range of about 1:4 or more to about 3:8 or less. Each layer of conductive material 574, 582, 590 has a length LP (e.g., 85 mm) in a range, for example, from about 70 mm or more (e.g., about 75 mm or more, about 80 mm or more) to about 100 mm or less (e.g., about 95 mm or less, about 90 mm or less, about 85 mm or less).

Each encapsulating or dielectric layer 570, 578, 586, 594 has a length LS (e.g., 90 mm), in a range, for example, from about 75 mm or more (e.g., about 80 mm or more, about 85 mm or more) to about 105 mm or less (e.g., about 100 mm or less, about 95 mm or less, about 90 mm or less). Each encapsulating or dielectric layer 570, 578, 586, 594 has a width WS (e.g., 7 mm) in a range, for example, from about 3 mm or more (e.g., about 3.5 mm or more, about 4 mm or more, about 4.5 mm or more, about 5 mm or more, about 5.5 mm or more, about 6 mm or more, about 6.5 mm or less) to about 11 mm or less (e.g., about 10.5 mm or less, about 10 mm or less, about 9.5 mm or less, about 9 mm or less, about 8.5 mm or less, about 8 mm or less, about 7.5 mm or less, about 7 mm or less).

While the terminating portion 618 of each layer of conductive material has a rectangular shape extending from a wider, rectangular shape of the layer 574 in FIGS. 8A and 8C, the portion 618a may be a different shape with different dimensions described above. While the portion 618b of the second layer of conductive material 582 is offset from the portions 618a, 618c, of the first and third layers of conductive material 574, 590, in other examples, the portion 618b configured to connect to an electrical lead may be disposed at an opposite end of the layered assembly 566 relative to the portions 618a, 618b.

This method may be used to fabricate layered sensors used to measure displacement for other applications unrelated to erectile function. In other examples, the dimensions provided above may be different depending on the subject or object the sensor is configured to measure.

In the illustrated example, the layers of conductive material 574, 582, 590 are layers of conductive ink. The conductive material may be metallic-based (e.g., silver, silver chloride, platinum, copper, gold, etc.), carbon-based (e.g., graphene or carbon nanotube-based), polymer-based (e.g., polymer thick film (PTF)), ionic gel paste-based, silicone-based, or combinations thereof. In other examples of the layered assembly 566, the layers of conductive material 574, 582, 590 may be tape, glue, paint, or combinations thereof.

The base layer 570 and the top layer 594 are encapsulating layers of an elastic material, and the first and second layers of dielectric material 578, 586 are also an elastic material. In the illustrated example, each of the encapsulating layers 570, 594 and dielectric layers 578, 586 of the layered assembly 566 is silicone. In other examples, each of the encapsulating layers 570, 594 can be polyurethane, or latex, or other natural or synthetic elastomeric materials.

The silicone material of the layered assembly 566 may have at least two durometers. For example, the first portion (e.g., first end) 598, 598a, 598b, 598c, 598d has a first durometer (e.g., 50A) and the second portion (e.g., second end) 606, 606a, 606b, 606c, 606d has a second durometer (e.g., 25A) less than the first durometer. The first durometer of the silicone material may be in a range, for example, from about 10 A or more (e.g., about 15 A or more, about 20 A or more, about 25 A or more, about 30 A or more) to about 60 A or less (e.g., about 55 A or less, about 50 A or less, about 45 A or less, about 35 A or less).

The setting portion 505 is a silicone material with a durometer (e.g., 50A) similar to the durometer of the first durometer of the encapsulating and dielectric layers of the layered assembly 566. The setting portion 505 provides a non-slip exterior surface to comfortably grip a skin surface of the user.

Like the wearable device 100 of FIG. 1, the wearable device 500 may be attached to a user before an erection session by placing the band 504 around the base of the penis 105b and adhering the housing assembly 502 to a skin surface of the user (e.g., the perineum, inner thigh, lower abdominal region, or other area).

FIG. 9A is a different example encapsulated sensor body 950 coupled to electrical connections 1024, 1028. The sensor body 950 is similar to the sensor body 550 described above and illustrated in FIGS. 8B and 8C. For the sake of simplicity, elements of the sensor body 950 in FIG. 9A that are similar to the elements of the sensor body 550 and electrical connection 506 are designated by the same reference numeral, incremented by 500. A description of many of these elements is abbreviated or even eliminated in the interest of brevity. In many respects, the body 550 and body 950 capture capacitance values in the same or similar manner.

The sensor body 950 includes three conductive ink layers 974, 982, and 990 separately printed on elastic material layers 994, 986, 978, and 970. Each electrical lead 1024, 1028 is connected to at least one of the conductive ink layers 974, 982, and 990.

The electrical leads 1024, 1028 of the electrical connection are configured to connect to the PCB 520 in the housing assembly 502 of the wearable device. In the illustrated example of FIG. 9A, a first electrical lead 1024 providing an electrical current is connected to conductive layer 990 and layer 974, and a second electrical lead 1028 for receiving an electrical current is connected to the conductive layer 982.

A circuit 930 representing the layered sensor body 550 of FIG. 8C and 950 in FIG. 9A functions similarly to a parallel plate capacitor. In FIG. 9B, components of the example sensor body 550 (coupled to electrical connection 506), 950 are illustrated as a circuit diagram. While the conductive ink layers 990, 982, and 974 are approximately parallel, e.g., may or may not be exactly parallel, the circuit 930 is a simplification to demonstrate how the sensor 950 can be used to measure capacitance.

As depicted in the circuit 930, a first electrical lead 1024 can supply an electric current from alternating current (AC) power supply 935. For example, the power supply 935 can be disposed in the housing 502 connected of the sensor body 950. In some cases, the AC power supply 935 supplies an alternating current in the range of 6-350 μA. In particular, the AC power supply 935 can supply an excitation signal, e.g., a square wave or sine wave, through the electric lead 1024 or the electric lead 1028.

For illustrative purposes, the conductive layers 990, 974 are connected to the first electrical lead 1024, e.g., the layers 574, and 590 of FIGS. 7B and 8A-8C, and are represented in the circuit 930 as positive plates of a parallel plate capacitor. Likewise, the conductive layer 982, e.g., the layer 582 of FIGS. 7B and 8A-8C, connected to the second electrical lead 1022 is represented in circuit 930 as the negative plate of a parallel plate capacitor. Generally, the elastic material can be an insulating material, and therefore function as a dielectric layer between the positive and negative plates. In more detail, as current flows positive charge builds up on the conductive layers 990, 974 and negative charge builds up on the conductive layer 975, resulting in the layers 990, 974 functioning as the positive plates of a parallel plate capacitor, and the layer 974 functioning as the negative plate of a parallel plate capacitor.

In the example depicted, a single parallel plate capacitor is formed from the electric fields established between the adjacent positive and negative plates. More specifically, since the conductive layers 974, 982, and 990 do not extend to the edge of the sensor body 950, the connected elastic material functions as a single dielectric between conductive layer 990 and conductive layer 982 and between conductive layer 974 and conductive layer 982. Thus, the first and second conductive layers 990 and 982 and the elastic layer 986 between the conductive layers 990 and the second and third conductive layers 974, 982 and the elastic layer 978 between the conductive layers 974, 982 form a single parallel plate capacitor. In this context, the two positive layers sharing the negative layer increases, e.g., doubles in the case that the conductive layers are uniform and cover the same area on the sensor body 950, the area between the positive and negative plates of the single parallel plate capacitor, as will be described in more detail below.

In this context, the ability of the layered sensor body 950 to store electric charge in the dielectric elastic layer between the conductive ink layers is referred to as the capacitance. The capacitance C provided by the layered sensor body 950 can be described based on the formula:

$$C = ( ε ) * A / d$$

where A is the area shared by the conductive ink layers in the capacitor, d is the distance between the plates, and the dielectric constant F, which measures a material's ability to store electrical energy in an electrical field, can be determined based on the composition of the elastic material between the conductive ink layers.

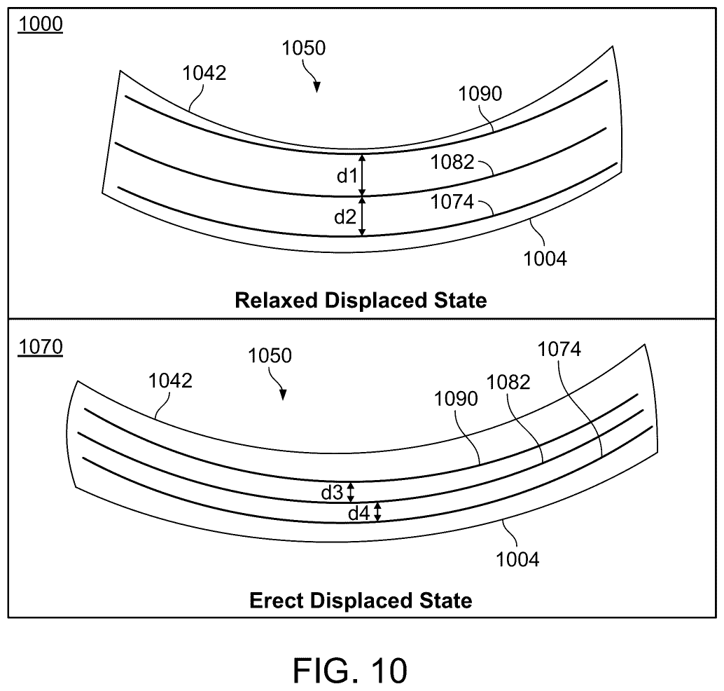

Since there is an inverse relationship between the distance between the positive and negative plates of the capacitor and the capacitance between the plates, the layered sensor is configured to measure a change in capacitance based on a displaced state of the wearable device. More specifically, the layered body 950 of FIG. 9A can be used to measure capacitance as the layered sensor body 950 is stretched to one or more displaced states of the wearable device, e.g., the wearable device 500. An example illustration of how displacement can be used to measure a change in capacitance based to a change in distance between the conductive ink layers as is depicted and described in more detail in FIG. 10.

However, the single parallel plate capacitor can be understood in this simplification as functioning as two parallel plate capacitors 944, 955 connected in parallel. The view shown in FIG. 9A illustrates how the first and second conductive layers 990 and 982 and the elastic layer 986 between the conductive layers 990, 982 can be treated as a first capacitor 944, and second and third conductive layers 974, 982 and the elastic layer 978 between the conductive layers 974, 982 can be treated as a second capacitor 955.

In the particular example depicted, the capacitance value is measured using a read-out circuit 960 that is coupled to the body 950. For example, the read-out circuit 960 can include a charge amplifier, charge-to-voltage converter, or charge integrator that can be used read-out the measure of capacitance through a series connection to the circuit 930. In particular, the read-out circuit 960 can be coupled to the sensor body 950 through analog front-end circuitry connected to the electrical connection.

In some cases, the read-out circuit 960 can be connected to the circuit 930 to measure the voltage or frequency of the displacement current flowing through the capacitor, e.g., after a supplied excitation signal with a known frequency and voltage. In this case, the read-out circuit 960 can indirectly measure the displacement current flowing through the capacitor by measuring the voltage across one or more known resistors connected in series to the capacitor (e.g., and included in the read-out circuit 960). The read-out circuit 960 can measure the voltage across the capacitor and use the voltage and the amplitude of the displacement current flowing through the capacitor to determine the total impedance of the circuit. Since the resistive impedance (resistance) of the resistor(s) is known, the read-out circuit 960 can calculate the capacitive reactance of the capacitor from the total impedance. The read-out circuit 960 can then use the capacitive reactance and the known frequency of the excitation signal to determine the capacitance.

FIG. 9C depicts an equivalent circuit diagram 980 of the circuit diagram of FIG. 9B. For example, the circuit 980 represents a simplified circuit having the same or similar electrical characteristics as the circuit 930. In this case, the simplification of the equivalent circuit 980 illustrates how the capacitors 944, 955 formed by the sensor body 950 can be represented as a single capacitor 998 with an equivalent capacitance.

As an example, in the case of a layered sensor with three conductive ink layers, e.g., the sensor body 550 of FIG. 8C and 950 of FIG. 9A, the equivalent capacitance of capacitor 944 and capacitor 955 in parallel can be calculated from the formula:

$${C}_{e q} = {C}_{1} + {C}_{2}$$

where C₁ is the capacitance for the capacitor 944 and C₂ is the capacitance for the capacitor 955. That is, C_{eq} is the equivalent capacitance of capacitor 978.

Generally, the equivalent capacitance of a layered sensor with N layers can be described as:

$${C}_{e q} = {\sum }_{i}^{\frac{N + 1}{{2}^{}}} {C}_{i}$$

where C_{eq} is the equivalent capacitance of the layered sensor based on the sum of the N capacitors formed by the sensor.

For example, a layered sensor with three conductive ink layers can capture a capacitance within the range of 70-240 pF. When the conductive layers are uniform, C1 and C2 are effectively equivalent, thereby reducing the calculation to a doubling of the capacitance of a single capacitor. Thus, returning to the single capacitor formed by the three conductive layers 972, 984, and 990 that can be understood as two parallel-plate capacitors functioning in parallel: since the equivalent capacitance of the parallel capacitors is a sum of the two capacitances of the capacitors 940 and 950 (with the same capacitance), the equivalent capacitance determined through the parallel-plate capacitors in parallel is the same as the capacitance of the single capacitor, where the shared negative layer 982 doubles the area.

While the circuit 930 described above with respect to FIGS. 9A-9C applies to a layered sensor with three conductive ink layers, generally, a layered sensor with two or more conductive ink layers may also be represented in a simplification as one or more parallel-plate capacitors in parallel. That is, the single parallel-plate capacitor with more conductive layers results in an increase in the area between the positive and negative plates of the single capacitor that can be understood as resulting in the equivalent capacitance of multiple parallel-plate capacitors in parallel.

For example, in a layered sensor with five conductive ink layers, the positive electrical lead may be connected to three of the five conductive layers, and the negative electrical lead may be connected to the remaining two of the five conductive layers, resulting in a simplified circuit with three parallel plate capacitors in parallel. As another example, in a layered sensor with seven conductive layers, the positive electrical lead may be connected to four of the seven conductive layers, and the negative electrical lead may be connected to the remaining three conductive layers, resulting in simplified circuit with four parallel plate capacitors in parallel.

FIG. 10 illustrates how an example layered sensor may be used to capture different capacitance values. FIG. 10 depicts a cross section of the sensor that exposes the base layer, e.g., without the top layer shown. More specifically, the view depicts the base layer 1042 and conductive ink layers 1090, 1082, and 1074 on the base layer 1042, as though the top layer has been pulled back from the assembly.

In this example, panels 1000 and 1070 depict the relative placement of the conductive ink layers of the band (e.g., 504 of wearable device 500) in two displaced states: a relaxed state (e.g., panel 1000) and an erect state (e.g., panel 1070). Similar to FIG. 7A, the band of a wearable device includes a layered body with three conductive ink layers. Elements of the sensor 1050 which are similar to the elements of the sensor body 550 are designated by the same reference numeral, incremented by 600. A description of many of these elements is abbreviated or even eliminated in the interest of brevity.

When the sensor body 1050 is in the relaxed displaced state shown in panel 1000, the sensor body 1050 still captures a displaced relative to an initial state when the wearable device is not being worn. In the relaxed displaced state, the conductive layers 1090, 1082 are spaced apart by distance d1, and the conductive layers 1082, 1074 are spaced apart by distance d2, resulting in a first capacitance value.

When the sensor body 1050 is in the erect displaced state shown in panel 1070, the wearable device is displaced corresponding with sexual stimulation of the penis of the user. In this illustration, the relative placement of the conductive layers in the device in panel 1070 are much closer together than the conductive layers in panel 1000. That is, as blood flows to the penis, the penis becomes more rigid, thereby transitioning the wearable device from the relaxed displaced state (e.g., panel 1000) to the erect displaced state (e.g., panel 1070).

As the penis become more rigid, the penis exerts a force against the interior surface 1042 of the band 1004, causing the layered body of the sensor body 1050 to stretch. As the layered body of the sensor 1050 stretches, the distance d1 and the distance d2 between the conductive layers 1090, 1082, and 1074 decreases. In the erect displaced state, the conductive layers 1090, 1082 are spaced apart by distance d3, and the conductive layers 1082, 1074 are spaced apart by distance d4, where d3 is less than d1 and d4 is less than d2, resulting in a second capacitance value.

Since the distance between the conductive layers is smaller in the erect displacement state, the capacitance value is higher, e.g., based on the inverse relationship described in detail with respect to FIG. 9B. Thus, the sensor 1050 can be used to measure different capacitance values based on the displacement of the band 1004.

While FIG. 10 depicts change in capacitance between two discrete displacement states, e.g., a relaxed and an erect state, the layered sensor 1050 may be used to measure capacitance values corresponding with a plurality of displaced states. The layered sensor 1050 is configured to capture a capacitance value at any point during use of the wearable device (e.g., during an erection session). With these captured values, a capacitance processing engine integrating the wearable device can use the one or more capacitance values captured during an erection session to determine an erection quality score of the user based on the change in capacitance corresponding with changes in displacement states.

FIG. 11 illustrates an example system 1100 for determining erectile function using the device 500 of FIG. 5. The system 1100 includes a capacitance processing engine 1106 that receives data from the device 500 for determining erectile function. The capacitance processing engine 1106 can generate displacement values that measure how much the band 504 of the device 500 is displaced by a volume of the penis 105b.

In general, the system 1100 obtains capacitance values 1104 from the device 500 and performs an action in response to processing the capacitance values 1104. In some examples, the system 1100 is included in a housing of the housing assembly 502. In some examples, the system 1100 may be communicably connected to one or more elements of the housing assembly 502, e.g., via a wired or wireless connection. The system 1100 may be used with other example wearable devices, but for ease of reference, the system 1100 will be described with reference to the device 500 of FIG. 5.

Capacitance values may be correlated with penis circumference and rigidity. For example, as a penis becomes more rigid, the circumference of the penis increases, exerting an outward force on the band 504 of the device 500, resulting the layered sensor body 550 stretching. As described with respect to FIG. 10, the expansion of the layered sensor body 550 of the device 500 increases the capacitance.

The capacitance processing engine 1106 can use one or more capacitance values 1104 obtained from the wearable device 500 at one or more displacement states of the device 500. More specifically, the system 1100 can generate displacement values based on how much a penis expands and remains rigid using the capacitance values 1104, which depend on how much the band 504 of the device 500 is displaced by a volume of the penis 105b. The system 1100 can use the displacement values to determine the erection quality score.

For example, the system 1100 can generate an erection quality score based on a discrepancy between one or more measured capacitance corresponding with a displaced state of the device 500, and a measured capacitance corresponding with the relaxed displaced state of the device 500, e.g., when the device 500 is worn without sexual stimulation. In particular, the relaxed displacement state of the device 500 can provide a baseline capacitance value that can be used for comparison with capacitance values corresponding with one or more displaced states resulting from an erection.

More specifically, the system 1100 can generate respective displacement values for each of a number of capacitance values 1104 corresponding with different displaced states of the device 500 and representing different circumferences of the penis 105b. As an example, the capacitance processing engine 1106 can receive the capacitance values 1104 as a sequence of data from the device 500 when the device is worn, e.g., during sexual stimulation of the penis 105b.

In some cases, the system 1100 can determine one or more correction factors for the erection quality score, e.g., using the values in the table. The system 1100 can scale the erection quality score using the correction factors, can reduce or increase the erection quality score using the correction factors, or both.

For example, the system 1100 can normalize the erection quality score based on each individual wearer's penis circumference. As an example, the capacitance values for men with a max girth of 11 cm will be distinct from those of 12 cm, 13 cm, etc. In some cases, the system 1100 can determine one or more correction factors for a maximally displaced state corresponding with the maximal girth of the penis 105b. In other cases, the system 1100 can determine one or more correction factors for a relaxed displacement state of the device 500 corresponding with a flaccid state of the penis 105b.

As another example, the system 1100 can standardize the erection quality score based on one or more calibration constants of the layered sensor. In this case, the system 1100 can obtain the calibration constants as an input, e.g., from another system. As an example, the layered sensor can be evaluated to determine the calibration constant(s) when it is manufactured. As another example, prior to being worn around the penis 105b, the system 1100 can determine the calibration constant(s).

In an example implementation, the erection quality score can be determined as: a*b(C_{measured}−C_{baseline}), where a is a correction factor for the baseline circumference of the penis 105b, b is a correction factor for the layered sensor, C_{measured} is the measured capacitance during sexual stimulation of the penis 105b and C_{baseline} is the measured capacitance during no stimulation of the penis 105b. In another example implementation, the erection quality score can be determined as (C_{measured}−C_{baseline})−c−d, where c is a correction factor for the baseline circumference and d is a correction factor for the layered sensor. In yet another example, implementation, the erection quality score can be determined as a*b(C_{measured}−C_{baseline})−c−d, where a is a first correction factor for the baseline circumference of the penis 105b, c is a second correction factor for the baseline circumference of the penis 105b, b is a first correction factor for the layered sensor, and d is a second correction factor for the layered sensor.

In some cases, the system 1100 can use a table of values to generate an erection quality score. The one or more values within the table of values can include band resistance factors, maximal girths of a particular individual or type of individual, associated quality scores determined with previous collected data, or other data associated with a particular quality score. In more detail, the table of values can include one or more values associated with a predetermined quality score. In this context, a predetermined quality score can be a quality score assigned at a particular combination of values in the table by a medical provider. In some cases, the predetermined quality scores were determined by a person using the wearable device 500. In other cases, the predetermined quality scores were determined using by a medical provider using a different measurement device.

In particular, the values can function as reference values, and the system can use the reference values to determine an erection quality score. That is, the system 1100 can generate an erection quality score based on a comparison of one or more detected displacement values, e.g., calculated from resistance values, capacitance values, etc., for a given penis and one or more values within the table of values that are associated with predetermined quality scores.

For example, the system 1100 can maintain one or more tables of values, and the system 1100 can identify a particular table for a particular individual, or type of individual, based on the maximum circumference of a given penis for use in calculating quality scores for the individual. Each table can correspond with different ranges of maximum erection circumferences. Based on the individual, the system 1100 can identify a table corresponding with a range of maximum circumferences including the maximum circumference of the given penis to provide one or more values associated with the quality score.

In some cases, the system 1100 can generate an erection quality score using the table by identifying and matching one or more values in the table to identify a corresponding predetermined quality score. For example, if the one or more detected displacement values and the one or more table values match, the system 1100 can generate an erection quality score that matches the quality score provided by the table. In other cases, the system 1100 can generate an erection quality score using the table by scaling between or aggregating existing values in the table. For example, if the one or more detected values and the one or more table values do not match, the system 1100 can generate an erection quality score that is based on predetermined quality scores by applying a scaling function between the existing values in the table that correspond with the predetermined quality scores, e.g., linear scaling, exponential scaling, quadratic scaling, among others. In some cases, the linear scaling can be a weighted average, e.g., between two predetermined quality scores based on the weighting of one or more values used to identify the corresponding quality score in the table.

An erection quality score can include an indication of rigidity of a penis. In some cases, the system 1100 determines rigidity using one or more previously measured factors of girth, known resistive forces of the penis received or sensed by (or imparted onto or against) the wearable device, and actively-monitored displacement. For example, the system 1100 can be configured to determine a rigidity of an erection by obtaining the capacitance values 1104 and using the table. The system 1100 can use one or more capacitance values 1104 to determine a penis circumference over time. The system 1100 can use resistive forces, such as a resistive force of the penis received or sensed by (or imparted onto or against) the wearable device 500 at the one or more displacement values to determine the rigidity of the penis. More specifically, the system 1100 can determine the resistive force using the displacement values and a known resistive factor of the elastic material in the band 504 worn on the penis at each of a number of time steps to help determine rigidity, circumference values, or quality scores. For example, the system 1100 can determine the resistive force at a particular time step by multiplying the resistance factor of the band 504 by the circumference of the penis at the particular time step, where the circumference of the penis is determined using the displacement value (e.g., resistance value, capacitance value, etc.) at the particular time step.

In some cases, previous factors of girth, such as a maximum circumference, can be compared with current circumference measurements to determine a personalized indication of a quality of an erection. As an example, the system 1100 can generate a rigidity score characterizing a rigidity of a penis by identifying a table corresponding with the maximum circumference of the given penis and determining the discrepancy between the current displacement of the penis, as measured by the detected displacement values, and the maximum displacement of the penis based on the maximum circumference of the given penis.

The system 1110 can then use the discrepancy between displacement values and the resistive force received by the band 504 of the wearable device 500 at the current displacement value to determine the rigidity score. More specifically, the system 1100 can determine the amount of resistive force received or sensed by the band 504 based on the real-time circumference of the penis and the resistance factor of the band 504 to enhance the robustness of the calculation of rigidity. The system 1100 can determine how the discrepancy between the current displacement and the maximum displacement correlates with the rigidity based on the resistive force, e.g., over one or more time steps. By associating the rigidity score with the resistive force of the band 504, the system 1100 can provide for a robust rigidity score that is normalized by both maximum circumference of a given penis and relative to the resistive force received or sensed by (or imparted onto or against) the band 504.

While described above relative to determining a rigidity score, resistive forces of the band 504 of the wearable device 500 can generally be used to improve accuracy of determined values, such as determined values of circumference or quality scores. In general, a circumference closer to a maximum circumference, maintained for longer, and where a band has a higher resistive force, can indicate an erection that is more rigid or of higher quality than a circumference further from a maximum, maintained for a shorter time, and where a band has a lower resistive force.

The system 1100 may be embodied in one or more circuit elements disposed in the housing assembly 502, such as the PCB 520 of the housing assembly 502. The system 1100 may be embodied in one or more computers, e.g., one or more computers communicably connected to the device 500. One or more computers may include a personal electronic device, such as a smartphone or laptop, that is configured to receive signals indicating the capacitance values 1104 or a processed result of the system 1100.

The capacitance processing engine 1106 obtains the capacitance values 1104 from the device 500. The capacitance values 1104 indicate an ability of the sensor assembly 501 of the device 500 to store electric charge. The capacitance values 1104 may be processed and graphically displayed.

For example, items 1108 and 1110 show example graphs of different individuals showing how capacitance values may change over time during an erection session. In the graphs, the x-axis is time and the y-axis can be an indication of penis circumference, such as a percentage of maximum known girth, e.g., where 100% corresponds to an erection that matches a maximum known girth. As an example, the capacitance value captured at a particular time can be used to determine a displacement value, which can be used to determine the circumference of the penis at the particular time.

Graphs can be provided to a user. Graphs can include any appropriate measurements for the y-axis that indicate an erection, such as length measurements, resistance values, % of maximum girth, among others.

Item 1108 is indicative of an older individual with severe erectile dysfunction. The graph of item 1108 shows results of an individual who can achieve a moderate erection, but not maintain, the erection during an erection session. The device 500 records a medium peak 1108a corresponding to an erection that is not strong and does not last very long, e.g., less than a minute. This data may be provided to a user, such as the individual, medical professional, or other, to improve diagnostic or treatment plans. Item 1110 is indicative of a younger individual who has a penis with wide girth, but is unable to achieve a strong erection. The graph of item 1110 shows results of an individual who does not achieve even a moderate erection during an erection session, recorded as a plateau 1110a. Again, this data may be provided to a user, such as the individual, medical professional, or other, to improve diagnostic or treatment plans. Items 1108 and 1110 are example interpretations of data obtained via the device 500.

In some cases, the capacitance processing engine 1106 includes one or more engines, such as subengines. The one or more engines can perform one or more operations based on the capacitance values 1104. For example, an erection identifier engine 1106a identifies one or more erections in one or more erection sessions. An erection quality score engine 1106b can determine one or more quality scores of one or more erections. A connected device engine 1106c can provide one or more data signals to connected devices, such as an activation signal 1114 to a light 1116 configured to turn on the light 1116 or data indicating an erection quality score 1118 provided to a personal electronic device 1120.

In some cases, the capacitance processing engine 1106 determines a start of an erection session using the obtained capacitance values 1104. The capacitance values 1104 can include noise or other values that were not generated based on an erection, but were instead generated, for example, when a user first places the device 500 around the penis, adjusts a fit, or other action that affects capacitance values. The capacitance processing engine 1106 can process the obtained capacitance values 1104 and determine whether a subset of values indicates an erection session or indicates noise or other non-erection actions, such as, for example, stretching the band while adjusting or putting on the device 500.

When the device 500 is worn by a user during sexual activity, the device 500 is configured to transmit, via a wired or a wireless connection, the capacitance values 1104 to the capacitance processing engine 1106. In some cases, the capacitance identifier engine 1106 identifies a start of an erection session, e.g., a time period when a penis satisfies a threshold circumference or capacitance value. For example, the erection identifier engine 1106a can use a set of one or more capacitance values to determine that an erection session started at a time corresponding to the one or more values. The erection identifier engine 1106a can include various processing to determine a start of an erection session, such as capacitance or time thresholds. For example, the erection identifier engine 1106a can determine whether a given capacitance value, or value range, is measured over a threshold amount of time. If the value, or value range, is maintained and satisfies a threshold time, the erection identifier engine 1106a can identify a corresponding start of an erection session.

In some cases, in response to identifying an erection session, the erection identifier engine 1106a can remove one or more capacitance values from memory. For example, the erection identifier engine 1106a can be connected to a memory device configured to store the capacitance values 1104. An erection session can be identified by a subset of capacitance values. After identifying an erection session, e.g., at the start of an erection session or subsequently in post processing scenario, the erection identifier engine 1106a can delete the subset of capacitance values that are not indicative of the erection session. In this way, the erection identifier engine 1106a can help reduce storage requirements, e.g., by removing from memory data that is not indicative of erection sessions.

In some cases, the erection identifier engine 1106a determines a start of an erection session based on capacitance values satisfying a threshold. For example, at least a subset of the capacitance values 1104 can indicate a penis circumference that satisfies a threshold circumference. The threshold circumference can be a static value for all users or can be user-specific. For example, a threshold circumference can be provided using an input interface where a user provides a circumference or can be determined based on user data, e.g., a user specifying a time period where an erection occurred or a pre-measured baseline value. In some cases, the baseline value can be determined based on input from a medical provider.

In some cases, the erection identifier engine 1106a includes one or more models (e.g., machine learning models) that learn to identify an erection session based on obtaining ground truth data from a user indicating when an erection session occurred, e.g., a time range, start, or finish. The erection identifier engine 1106a can provide capacitance values and other related values, e.g., from the table described above, as input data to one or more models. The erection identifier engine 1106a can compare output of the models to one or more ground truth data items indicating one or more erection sessions. Based on error between the output of the models and the ground truth data, the erection identifier engine 1106a can update the one or more models, e.g., by updating one or more parameters or layers of one or more models.

In some cases, the erection quality score engine 1106b determines an erection quality score of an erection session. The erection quality score engine 1106b can estimate a current circumference of the penis measured by the device 100 and a previously stored circumference of the penis to generate a quality score. In some cases, the stored circumference can indicate a maximum circumference of the user—e.g., measured using the device, calipers, fabric ruler, string, or other measurement tool by the user.

For example, the erection quality score engine 1106b can determine a current circumference of the penis using the capacitance values 1104, e.g., by determining a displacement value and adding the displacement value to a baseline circumference for the penis 105b of the user. In this context, a displacement value indicate an amount that a volume of the penis 105b displaces the band 504 of the device 500. Using the one or more displacement values, the erection quality score engine 1106b can generate one or more erection quality scores.

In more detail, the erection quality score engine 1106b can compare a current circumference, indicated by the obtained capacitance values 1104, to the stored circumference to determine a personalized quality score for an erection. For example, a person with a small penis may have an erection with a circumference of X inches. The quality score of this erection compared to a larger penis with a maximum circumference of Y (where Y>>X) would be poor but, compared to the maximum circumference (e.g., a baseline value) of the smaller penis, the quality score may be higher. Quality scores can be represented in any suitable alphabetic, numeric, alphanumeric, color codes, or other indications. In some cases, a quality score includes a ratio of a current penis circumference over a maximum circumference (e.g., baseline value) for the same penis. A quality score can be measured throughout an erection session or calculated as an average, e.g., by comparing an average session circumference with a maximum circumference or by comparing a maximum session circumference with a previously stored maximum.

In some cases, the connected device engine 1106c performs an action based on the processing of one or more capacitance values. For example, the connected device engine 1106c can provide an indication of the erection quality score to a user. The erection quality score 1118 can be indicated via a transmitted signal configured to display the erection quality score 1118 on a display of a device, such as the personal electronic device 1120. A quality score can be a number or a set of numbers indicating a quality score at different points of time within an erection session or can indicate an average quality score for a session. The connected device engine 1106c can transmit an indication of one or more quality scores to one or more devices in real-time—e.g., during a sexual activity that causes an erection session to be measured, the connected device engine 1106c can provide indications to one or more devices. Indications can include values, graphs, charts, images, or other displays.

In some cases, providing an indication includes providing an indication during sexual activity. The system 1100 can generate and provide output to a device, such as the device 1120 which can include a smartphone, laptop, or other device, information that can be useful to a wearer or a sexual partner of the wearer. Information can include updates on one or more of a current erection session, quality scores, motivational messages, information on goal achievement, suggestions for changing sexual positions or activities, etc. Information can be provided via audio, visual (e.g., display, lighting), or tactile (e.g., vibration of the device) feedback.

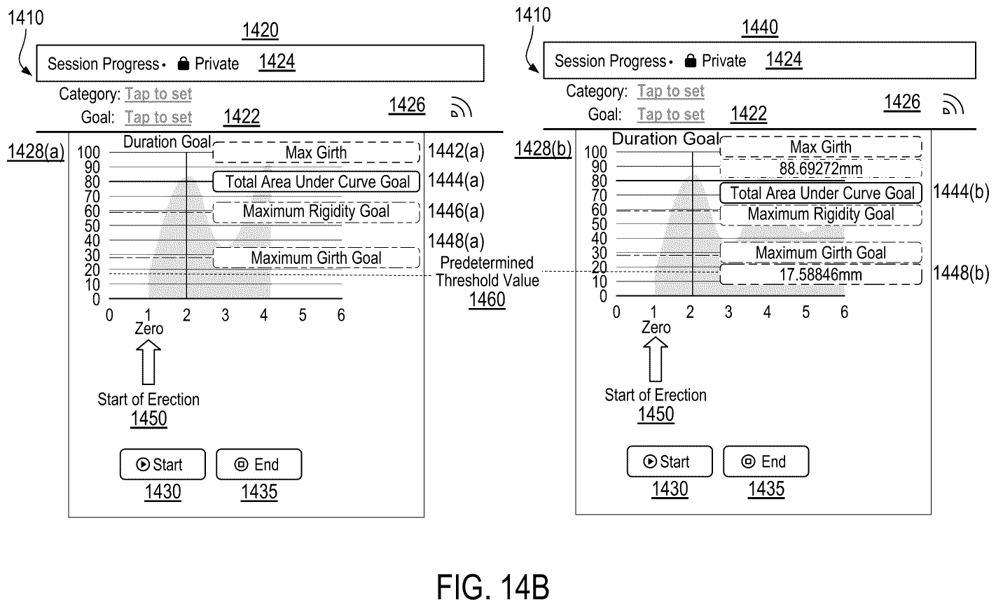

Goals for erectile function can be set by a user before sexual activity begins, e.g., in consultation with a medical professional or during setup of an application that pairs with the device 500. The system 1100 can be used to provide updates on these goals in real time, including providing specific feedback tailored to the user, e.g., motivational indications, critical indications, notes for sexual adjustments based on sensed data of a current sexual activity using one or more sensors, among others. Example user interfaces included in a user flow for setting a goal and receiving feedback tailored to the goal will be described in more detail with respect to FIGS. 14A-14B.

In some cases, multiple erection sessions can be combined for providing feedback, such as trending or historical data. For example, the system 1100 can calculate averages or other metrics based on one or more sessions. The system can generate various indications, such as overlaid plots or comparisons of times for one or more sessions. Times can indicate a time it takes to achieve an erection for each session or a time an erection was maintained. Feedback can include a summary of one or more sessions. Summaries can be generated using one or more trained models, such as large language models. Feedback can include questions for a user, such as psychological questions to help gauge one or more psychological factors that could impact erectile performance. Feedback can include notifications to recalibrate a sensor, e.g., after a detected change in average size of a penis caused by growth or a change in user.

In some cases, the connected device engine 1106c activates one or more devices. For example, the connected device engine 1106c can transmit the activation signal 1114 to a light 1116. The light 1116 can communicate with an IoT device or a device with an element for receiving a signal from the system 1100. The light 1116 can be configured to activate a light in response to receiving the activation signal 1114. The connected device engine 1106c can generate the activation signal 1114 to cause the light 1116 to change colors, change intensity, switch on or off, or a combination of these among others.

In some cases, the capacitance processing engine 1106 compares one or more generated quality scores to one or more stored erection quality scores. For example, the capacitance processing engine 1106 can compare data, such as erection quality scores, of a user with the same user or other users. For quality scores, comparison with other users can indicate whether a current user is having higher or lower quality erection scores compared to a population, such as other users of a device or other pools of data. Comparison data can include local, on device data, or cloud data.

In some cases, the capacitance processing engine 1106 determines a series of two or more circumference measurements of a penis. For example, during one or more erection sessions, the capacitance processing engine 1106 can determine two or more circumferences based on one or more electrical capacitance values. In some cases, the capacitance processing engine 1106 determines a measure of rigidity using two or more determined circumferences. For example, the capacitance processing engine 1106 can calculate an average rigidity based on two or more determined circumferences over time. Rigidity can be equal to area under a curve formed by circumference measurements over time.

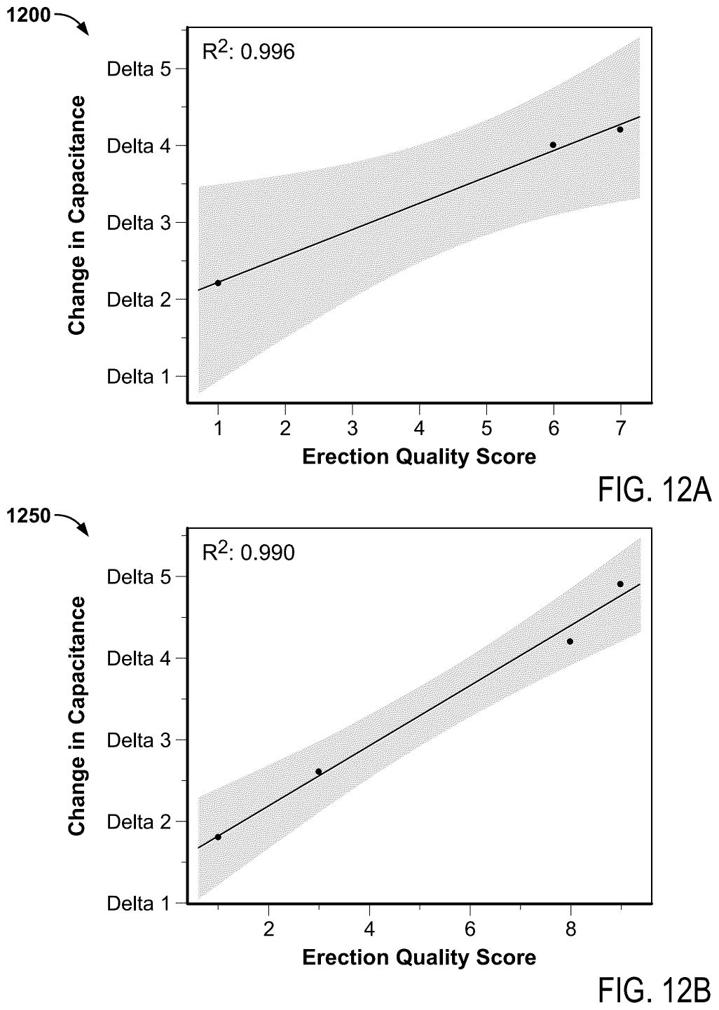

FIGS. 12A and 12B depict example results from a layered sensor, demonstrating the relationship between change in capacitance and erection quality score. As an example, the results depicted in FIGS. 12A and 12B can correspond with the layered sensor described with respect to FIG. 9.

Graphs 1200 and 1250 illustrates the relationship between change in capacitance between a baseline capacitance and a capacitance measured during sexual stimulation of the penis for two individuals. Graph 1200 depicts results for three erection sessions of individual A, and graph 1250 depicts results for four erection sessions of individual B.

In this case, each point in the graphs 1200 and 1250 represents a medical provider determined erection quality score for a particular erection session, and is plotted against the change in capacitance measured during sexual stimulation of the penis with respect to a baseline capacitance for the penis. As an example, the capacitance during sexual stimulation of the penis can be selected as the maximal capacitance measured during the erection session.

In particular, graphs 1200 and 1250 demonstrate the predictive power of the change in capacitance in predicting the erection quality score for individual A and B. That is, the lines of best fit determined using the change in capacitance as a regression variable for both individual A and for individual B are found to replicate the medical provider recorded erection quality score for the individuals A and B with high fidelity. Thus, the change in capacitance captured using a wearable device including a layered sensor between a baseline capacitance and a capacitance during sexual stimulation of a penis can be used as a predictive variable in determining the erection quality score for an individual.

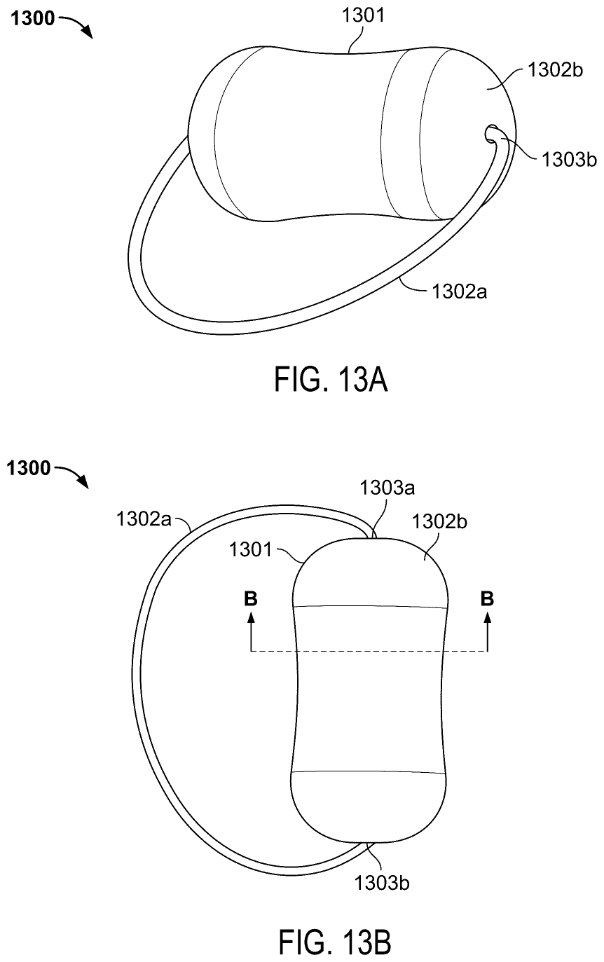

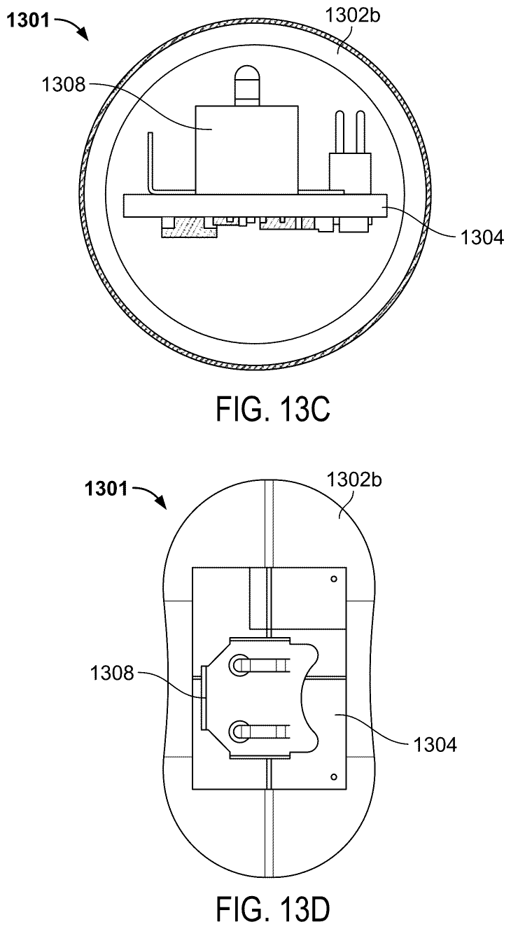

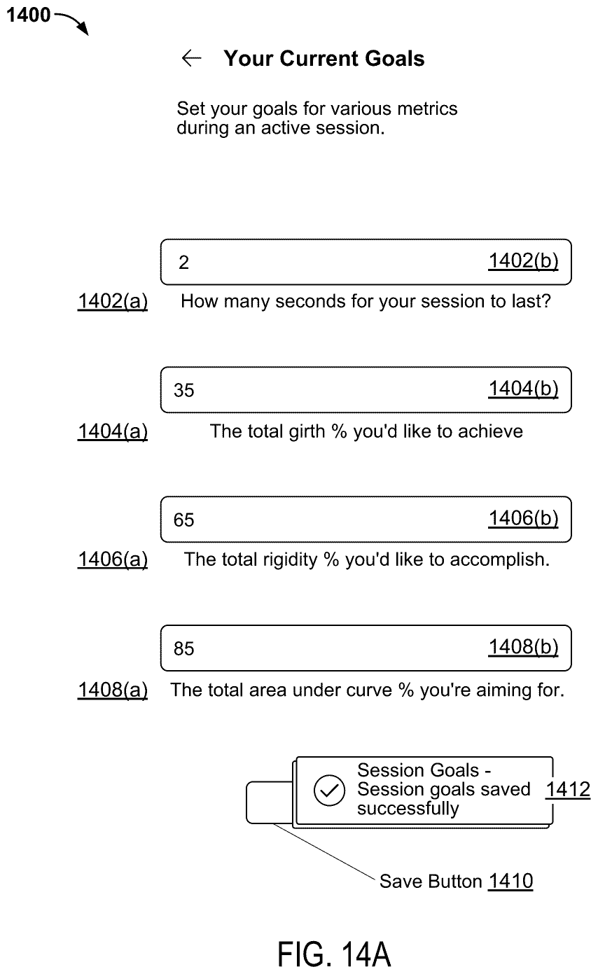

While the housing component 102 of the device 100 of FIGS. 1 and 2 is separated from the band 104 by the electrical connection 106, in other examples, a housing component of a wearable device for determining erection quality may be connected directly to a band, as shown in FIGS. 13A and 13B.

The device 1300 for determining erectile function can be used with the system 300 of FIG. 3, the system 1100 of FIG. 11 and any of the methods of FIGS. 4A-4C. A housing component 1302b of the device 1300 is disposed between opposite ends 1303a, 1303b the band 1302a. In some cases, the housing component 1302b rests against a shaft of a penis when the device 1300 is worn. The housing 1301 of the housing component 1302b is sealably coupled to the first and second ends 1303a, 1303b of the band 1302a to keep moisture and other fluids from seeping into the housing component 1302b.

The band 1302a may be selected of several bands having varying lengths, e.g., depending on a size of a penis to be measured.

In FIGS. 13C and 13D, the housing 1301 surrounds and protects a PCB 1304 and battery support 808 from the environment. In general, a thinner housing may reduce weight at the expense of decreased structural integrity. The circuit board 1304 includes connections to provide or measure electrical current through the band 1302a. Such measurements can be used to generate resistance values, such as the resistance values 304 of FIG. 3. One or more circuits for processing or generating measuring data are housed within the housing 1301.

In some cases, housings are made substantially flat, e.g., as shown in FIG. 1, to facilitate adhesion to a body of a user with minimal protrusion. In general, the width of a housing can be at least the width of a housed circuit board and the height of the housing can be at least the height of the housed circuit board. In general, a circuit board can include any number of circuit elements that may or may not include a central “board” with which circuit elements are affixed.

In some cases, the housing component may be positioned adjacent to or within a ring at least partially formed by the band when the band is worn on a penis. In some cases, the housing component may be positioned away from the band, e.g., to limit stretching of the band caused by jostling of the housing component.

In some cases, the housing component of the wearable device may be other shapes. For example, housings can be in any suitable shape, e.g., sufficient to at least partially enclose a circuit board for generating and reading electrical currents. In some cases, other types of fasteners are used to fasten the wearable device to an individual. For example, a belt, or other loop may be used to fasten a housing component to a human, e.g., worn around a thigh, ankle, neck, waist, or other body part. In some cases, a device is not attached to a body. For example, a housing component may sit on a bed, table, wall, or other area during sexual activity. In some cases, extension cables may be used to provide more room for positioning a housing component.

FIGS. 14A and 14B depict example user interfaces for use in a software application (“app”) on a user device coupled to the wearable device.

In some cases, the system can provide feedback to the wearer of the wearable device or other person to inform the individual of sexual performance by way of a user interface displayed on the display of a user device. For example, the feedback can be visual feedback with respect to predefined sexual-related goals.

FIG. 14A depicts an example user interface for setting sexual-related goals. For example, the user interface 1400 can be displayed in response to a selection of a menu option including a goal setting option on a home page of the app.

The user interface 1400 includes a visualization of multiple goal-related questions, with each goal-related question corresponding with an input portion for a user to enter in their goals. For example, the questions 1402(a), 1404(a), 1406(a), and 1408(a) can have been determined using input from a medical provider.

In the particular example depicted, a user can enter their respective goals in each of the input portions 1402(b), 1404(b), 1406(b), and 1408(b) corresponding with each question. In this case, the input portions 1402(b), 1404(b), 1406(b), and 1408(b) are implemented as text fields, and a user can enter their answer using a keyboard after selection each input portion 1402(b), 1404(b), 1406(b), and 1408(b). In another case, the input portions 1402(b), 1404(b), 1406(b), and 1408(b) can be implemented as a drop-down menu.

The user can then submit their goals using a save button 1410. In some cases, in response to the selection of the save button 1410, the user interface 1120 can provide a message that indicates whether the goals were successfully saved.

As an example, a user can use the user interface 1400 multiple times to, e.g., update their goals in accordance with progress in a course of treatment plan. As another example, in some cases, a user can save different goals for different purposes, e.g., corresponding with different types of sexual stimulation. In this case, the user interface 1400 can additionally include a label field to allow the user to label each goal, e.g., for retrieval when the app provides feedback.

FIG. 14B depicts example user interfaces for tracking an erection at a first and a second, later time during an erection session using a device for determining erectile function.

User interface snapshot 1420 and user interface snapshot 1440 are examples of a tracking user interface 1400 at two different times that illustrate how feedback can be provided to a user during an erection session. For example, an initialized tracking user interface can be displayed in response to a selection of a menu option including a tracking option on a home page of the app.

As an example, a user can select the tracking option and can select a goal for comparing tracked data to. As described with respect to FIG. 14A, in the case that multiple goals are saved, a user can indicate the goal that they wish to evaluate the erection session relative to, e.g., in the input portion 1422. In this case, the user did not select a goal. For example, in the case that only one goal has been saved using the user interface 1400 of FIG. 14A.

The user can select a start session button 1430 after putting on the wearable device. When the wearable device records the start of an erection 1450, e.g., based on a predetermined threshold value 1460, the user interface 1420 can populate a graph with data received by way of the wearable device. For example, the user device can be coupled with the wearable device, and can receive the data through a network transmission communication protocol, e.g., Bluetooth, or the Internet. In this case, the user is receiving the data by way of an internet connection, e.g., as indicated by the WiFi icon 1426.

While not depicted in the user flow presented, the user can terminate the tracking session by selecting an end session button 1435. As an example, after terminating the session, data is no longer provided by way of the tracking user interface 1400.

In the particular example depicted, the data is displayed as a graph that is normalized in terms of a user's maximum girth. That is, the graph displays the percentage of maximum girth achieved throughout the course of the erection session. For example, at the time corresponding with the user interface snapshot 1420, the graph 1428(a) includes values for the percentage of maximum girth achieved up to a first time.

The user interface 1400 can update as additional data is received from the wearable device. As a related example, at the later time corresponding with the user interface snapshot 1440, the graph 1428(b) includes more values than the graph in 1428(a). In some cases, data can be provided by way of the user interface 1420 or 1440 in near-real time, e.g., through streaming. In particular, each data point can be rendered on the display of the user device as the data is received, e.g., so the graph appears to be plotted from a set of values received in a sequence.

In this case, the graphs 1428(a) and 1428(b) are presented on an interactive graphical user interface (GUI). For example, as the data is being presented, or after the session has concluded, the user or another individual, e.g., can select any of the goal labels 1442(a), 1444(a), 1446(a), and 1448(a) overlayed with the graph to view the value the individual achieved during the session corresponding with the goal. As an example, in response to a user selecting the minimum girth goal 1448(a), the graph GUI can present the value 1448(b) for the maximum girth the individual achieved during the session. As another example, in response to a user selecting the total area under the curve goal 1444(a), the graph GUI can present the value 1444(b) for the total area under the curve achieved by the individual during the session.

In the case of the total area under the curve goal, a processing engine that is executing the app in order to display the user interfaces 1420 and 1440 on the user device can fit or approximate a curve and compute the integral of the curve fit to the plotted values to determine the total area under the curve as the data is being displayed by way of the tracking user interface 1400. As an example, the total area under the curve goal can be approximated using a Riemann sum, or the trapezoidal rule. Since the calculation of the area under the curve can be computationally expensive relative to plotting received data points, the total area under the curve can be determined at a predefined interval, e.g., every 2 seconds, instead of when each data point is received and plotted.

In the particular example depicted, the erection session is private, e.g., as is indicated by the privacy indicator 1424. In this case, the user can have elected to not share data from the session with another individual, e.g., the user's medical provider. However, in the case that the erection session is not private, the user interfaces 1420 and 1440 can be displayed on another user device, e.g., concurrently or after conclusion of the erection session.

In this specification the term “engine” is used broadly to refer to a software or hardware-based system, subsystem, or process that is programmed to perform one or more specific functions. An engine can be implemented as one or more software modules or components, installed on one or more computers in one or more locations. In some cases, one or more computers will be dedicated to a particular engine; in other cases, multiple engines can be installed and running on the same computer or computers. In some cases, an engine can refer to hardware circuit elements configured to perform one or more operations.

The subject matter and the actions and operations described in this specification can be implemented in digital electronic circuitry, in tangibly-embodied computer software or firmware, in computer hardware, including the structures disclosed in this specification and their structural equivalents, or in combinations of one or more of them. The subject matter and the actions and operations described in this specification can be implemented as or in one or more computer programs, e.g., one or more modules of computer program instructions, encoded on a computer program carrier, for execution by, or to control the operation of, data processing apparatus. The carrier can be a tangible non-transitory computer storage medium. Alternatively or in addition, the carrier can be an artificially-generated propagated signal, e.g., a machine-generated electrical, optical, or electromagnetic signal, that is generated to encode information for transmission to suitable receiver apparatus for execution by a data processing apparatus. The computer storage medium can be or be part of a machine-readable storage device, a machine-readable storage substrate, a random or serial access memory device, or a combination of one or more of them. A computer storage medium is not a propagated signal.

The term “data processing apparatus” encompasses all kinds of apparatus, devices, and machines for processing data, including by way of example a programmable processor, a computer, or multiple processors or computers. Data processing apparatus can include special-purpose logic circuitry, e.g., an FPGA (field programmable gate array), an ASIC (application-specific integrated circuit), or a GPU (graphics processing unit). The apparatus can also include, in addition to hardware, code that creates an execution environment for computer programs, e.g., code that constitutes processor firmware, a protocol stack, a database management system, an operating system, or a combination of one or more of them.

A computer program can be written in any form of programming language, including compiled or interpreted languages, or declarative or procedural languages; and it can be deployed in any form, including as a stand-alone program, e.g., as an app, or as a module, component, engine, subroutine, or other unit suitable for executing in a computing environment, which environment may include one or more computers interconnected by a data communication network in one or more locations.

A computer program may, but need not, correspond to a file in a file system. A computer program can be stored in a portion of a file that holds other programs or data, e.g., one or more scripts stored in a markup language document, in a single file dedicated to that program, or in multiple coordinated files, e.g., files that store one or more modules, sub-programs, or portions of code.

The processes and logic flows described in this specification can be performed by one or more computers executing one or more computer programs to perform operations by operating on input data and generating output. The processes and logic flows can also be performed by special-purpose logic circuitry, e.g., an FPGA, an ASIC, or a GPU, or by a combination of special-purpose logic circuitry and one or more programmed computers.

Computers suitable for the execution of a computer program can be based on general or special-purpose microprocessors or both, or any other kind of central processing unit. Generally, a central processing unit will receive instructions and data from a read-only memory or a random access memory or both. The essential elements of a computer are a central processing unit for executing instructions and one or more memory devices for storing instructions and data. The central processing unit and the memory can be supplemented by, or incorporated in, special-purpose logic circuitry.

Generally, a computer will also include, or be operatively coupled to, one or more mass storage devices, and be configured to receive data from or transfer data to the mass storage devices. The mass storage devices can be, for example, magnetic, magneto-optical, or optical disks, or solid state drives. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

To provide for interaction with a user, the subject matter described in this specification can be implemented on one or more computers having, or configured to communicate with, a display device, e.g., a LCD (liquid crystal display) monitor, or a virtual-reality (VR) or augmented-reality (AR) display, for displaying information to the user, and an input device by which the user can provide input to the computer, e.g., a keyboard and a pointing device, e.g., a mouse, a trackball or touchpad. Other kinds of devices can be used to provide for interaction with a user as well; for example, feedback and responses provided to the user can be any form of sensory feedback, e.g., visual, auditory, speech, or tactile feedback or responses; and input from the user can be received in any form, including acoustic, speech, tactile, or eye tracking input, including touch motion or gestures, or kinetic motion or gestures or orientation motion or gestures. In addition, a computer can interact with a user by sending documents to and receiving documents from a device used by the user; for example, by sending web pages to a web browser on a user's device in response to requests received from the web browser, or by interacting with an app running on a user device, e.g., a smartphone or electronic tablet. Also, a computer can interact with a user by sending text messages or other forms of message to a personal device, e.g., a smartphone that is running a messaging application, and receiving responsive messages from the user in return.

This specification uses the term “configured to” in connection with systems, apparatus, and computer program components. That a system of one or more computers is configured to perform particular operations or actions means that the system has installed on it software, firmware, hardware, or a combination of them that in operation cause the system to perform the operations or actions. That one or more computer programs is configured to perform particular operations or actions means that the one or more programs include instructions that, when executed by data processing apparatus, cause the apparatus to perform the operations or actions. That special-purpose logic circuitry is configured to perform particular operations or actions means that the circuitry has electronic logic that performs the operations or actions.

The subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of what is being claimed, which is defined by the claims themselves, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claim may be directed to a subcombination or variation of a subcombination.

Similarly, while operations are depicted in the drawings and recited in the claims in a particular order, this by itself should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method for determining erectile function of an individual, the method comprising:
obtaining, from a wearable device comprising a band configured to be worn on a penis of the individual during stimulation of the penis, one or more displacement values characterizing a measure of displacement of the wearable device by the penis; and
determining, using a resistance factor of the band, a maximal circumference of the penis, and the one or more displacement values, an erection quality score of the penis.

2. The method of claim 1, wherein the resistance factor corresponds to a force range of 0.5 N to 10 N.

3. The method of claim 2, wherein the force range is approximately 1 N to approximately 6 N.

4. The method of claim 1, comprising transmitting an indication of the erection quality score to a user device.

5. The method of claim 1, comprising generating a feedback signal indicating erection performance, wherein the erection performance is calculated using the erection quality score.

6. The method of claim 1, comprising transmitting an indication of the erection quality score to one or more user devices in real-time.

7. The method of claim 1, comprising comparing the erection quality score to one or more stored erection quality scores.

8. The method of claim 1, wherein determining the erection quality score of the penis comprises calculating a rigidity of the penis using the one or more displacement values.

9. The method of claim 1, comprising:
activating the wearable device by generating an activation signal using a user device; and
transmitting the activation signal to the wearable device.

10. The method of claim 9, wherein transmitting the activation signal to the wearable device comprises sending a short-range wireless communication to the wearable device.

11. The method of claim 1, wherein the wearable device comprises a capacitance sensing circuit, and
wherein obtaining the one or more displacement values comprises obtaining, by the capacitance sensing circuit, one or more capacitance values.

12. The method of claim 11, wherein the wearable device comprises:
a layered sensor comprising:
a first layer of conductive material;
a second layer of conductive material approximately in parallel with the first layer of conductive material;
a dielectric layer between the first and second layers of conductive material;
a top layer configured to define an exterior surface of the layered sensor; and
a base layer configured to define an interior surface of the layered sensor; and
wherein obtaining the one or more capacitance values representing the measure of displacement comprises:
obtaining, from the capacitance sensing circuit, a capacitance value that specifies an ability to store electric charge between the first and second layers of conductive material at a displaced state of the wearable device,
wherein the one or more capacitance values corresponds with the displaced state of the layered sensor of the wearable device.

13. The method of claim 12, wherein the layered sensor comprises:
a third layer of conductive material between the first and second layers of conductive material; and
a second dielectric layer between the third layer of conductive material and the first layer or the second layer of conductive material.

14. The method of claim 11, wherein determining the erection quality score of the penis comprises:
determining the erection quality score using a measure of change in displacement of the wearable device at a first displaced state and a second displaced state based on the one or more capacitance values.

15. The method of claim 1, comprising:
modifying the erection quality score based on one or more correction factors, wherein at least one of the correction factors corresponds with the maximal circumference of the penis.

16. A method of communicating a quality of an erection of an individual, the method comprising:
obtaining, from a wearable device worn on a penis of the individual during stimulation of the penis, one or more displacement values characterizing a measure of displacement of the wearable device by the penis;
determining whether the one or more displacement values satisfy a predetermined threshold; and
in response to determining that the one or more displacement values satisfy the predetermined threshold, controlling a connected device to perform an operation.

17. The method of claim 16, wherein controlling the connected device to perform the operation comprises controlling a light to change an illumination state.

18. The method of claim 17, wherein controlling the light to change the illumination state comprises changing a color of the light from a first color to a second color.

19. The method of claim 17, wherein controlling the light to change the illumination state comprises controlling an intensity of the light.

20. The method of claim 16, wherein controlling the connected device to perform the operation comprises controlling a speaker to emit audio.

21. The method of claim 20, wherein controlling the speaker to emit audio comprises controlling the speaker to indicate a quality of erection based on the one or more displacement values.

22. The method of claim 16, comprising determining, using the one or more displacement values, an erection quality score of the penis.

23. The method of claim 22, comprising comparing the erection quality score to one or more stored erection quality scores.

24. The method of claim 16, comprising determining, using the one or more displacement values, an average rigidity of the penis over time.

25. A method comprising:
obtaining, from a wearable device worn on a penis during stimulation of the penis, one or more displacement values including a first set of values and a second set of values;
determining, using the one or more displacement values, a start of an erection session corresponding to the second set of values; and
removing, from memory, the first set of values.

26. The method of claim 25, wherein determining the start of the erection session corresponding to the second set of values comprises:
determining the second set of values indicate a rigidity that satisfies a predetermined threshold.

27. The method of claim 25, wherein the second set of values characterizes a measure of displacement of the wearable device by the penis; and
determining, using the second set of values, an erection quality score of the penis.

28. The method of claim 27, wherein the wearable device comprises a band worn around the penis, the method comprising:
obtaining a resistance factor of the band; and
wherein determining the erection quality score comprises using the resistance factor to determine the erection quality score of the penis based on a resistive force received by the wearable device.

29. The method of claim 28, wherein the resistance factor of the band corresponds to a force in a range of approximately 1 N to approximately 6 N.

30. The method of claim 27, comprising transmitting an indication of the erection quality score to a user device.
